(19) United States
(12) Patent Application Publication
(10) Pub. No.: US 2004/0063665 A1
(43) Pub. Date: Apr. 1, 2004

(54) Substituted benzopyranones as telomerase inhibitors

(76) Inventors: Alberto Bargiotti, Via Mario Donati (IT); Luisella Bonomini, Via Nazionale dei Giovi (IT); Maria Menichincheri, Via Felice Casati (IT); Jurgen Moll, Localita San Bartolome (IT); Paolo Polucci, Via F. Sforza (IT); Chiara Soncini, Piazza Crivelli (IT); Ermes Vanotti, Via Giovanni Cimabue (IT)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/471,919

(22) PCT Filed: Mar. 12, 2002
(86) PCT No.: PCT/EP02/02783

(51) Int. Cl. A61K 31/695; A61K 31/353

(52) U.S. Cl. 514/63; 514/456

(57) ABSTRACT

The present invention relates to benzopyranone derivative, to methods for treating telomerase-modulated diseases, in particular cancers, with said derivatives, to a process for their preparation, to their use as medicaments and to pharmaceutical compositions comprising them.

### FIELD OF THE INVENTION

The present invention relates to methods for treating telomerase-modulated diseases, in particular cancer, to compounds that inhibit telomerase activity, to a process for their preparation, to their use as medicaments and to pharmaceutical compositions comprising them.

### BACKGROUND OF THE INVENTION

Cancer is one of the major causes of disease and the second leading cause of death in the western world. Most cancer patients still die due to metastatic disease. Despite the great increase in the knowledge and understanding of the regulatory mechanisms involved in the onset of malignancy, currently available treatments (including surgery, radiation and a variety of cytoreductive and hormone-based drugs, used alone or in combination, are still highly non specific and toxic to the patient, causing severe side effects including nausea and vomiting, hair loss, diarrhea, fatigue and ulcerations. These evidences indicate the need for new and more effective anti-cancer therapies.

Recently an understanding of the mechanisms by which normal cells reach the state of replicative senescence, i.e. the loss of proliferative capacity that cells normally undergo in the cellular aging process, has begun to emerge and in this respect telomerase appears to have a central role.

Telomerase is a ribonucleoprotein enzyme responsible in most eukaryotes for the complete replication and maintenance of chromosome ends, or telomeres, which are composed of repeated DNA sequences (in particular human telomeres are formed by 5′-TTAGGG repeats). Telomerase binds to telomeric DNA using as a template a sequence contained within the RNA component of the enzyme necessary for the addition of the short sequence repeats to the chromosome 3′ end (see Blackburn 1992, Annu. Rev. Biochem., 61, 113-129). In most human somatic cells telomerase activity cannot be detected and telomeres shorten with successive cell division: in fact actively dividing normal cells have the potential to lose 50-200 base pairs after each round of cell division, finally resulting in shortening of telomeres. Recently it has been hypothesized that the cumulative loss of telomeric DNA over repeated cell divisions may act as a trigger for cellular senescence and aging, and that regulation of telomerase may have important biological implications (see Harley 1991, Mutation Research, 256, 271-282). In fact in the absence of telomerase, telomeres shortening will eventually lead to cellular senescence by various mechanisms. This phenomenon, thought to be responsible for cellular aging, is termed the “mitotic clock” (see Holt et al. Nat. Biotechnol., 1996, 15, 1734-1741).

Telomerase activity is restored in immortalised cell lines and in more than 85% of human tumors, thus maintaining telomeres length stable (see Shay, J. W. and Bacchetti, S. Eur. J. Cancer, 1997, 33, 787-791). Thus in cancer cells having telomerase activity and where the malignant phenotype is due to the loss of cell cycle or growth controls or other genetic damage, telomeric DNA is not lost during cell division and telomers are maintained, thereby allowing the cancer cells to become immortal, leading to a terminal prognosis for the patient.

Telomerase inhibition can lead to telomere shortening in tumors and subsequent senescent phenotype (see Feng et al. Science, 1995, 269, 1236-1241). Moreover it has been recently shown (Hahn et al. Nature Med., 1999, 5, 1164-1170) that inhibition of telomerase activity by expressing in tumor cells a catalytically-inactive form of human TERT (TElomerase Reverse Transcriptase, the catalytic subunit of the enzyme) can cause telomere shortening and arrest of cell growth and apoptosis. In addition peptide-nucleic acids and 2′-O-MeRNA oligomers complementary to the template region of the RNA component of the enzyme have been reported to cause inhibition of telomerase activity, telomere shortening and cell death in certain tumor cell lines (see Herbert et al. PNAS, 1999, 96, 14276-14281; Shammas et al. Oncogene, 1999, 18, 6191-6200). These data strongly support inhibition of telomerase activity as an innovative, selective and useful method for the development of new anticancer agents.

Thus compounds that inhibit telomerase activity can be used to treat cancer, as cancer cells express telomerase activity, while normal human somatic cells usually do not express telomerase activity at biologically relevant levels (i.e., at levels sufficient to maintain telomere length over many cell divisions). Also telomere length in tumors is reduced compared with non-transformed cells giving the possibility of a therapeutic window (see Nakamura et al. Cancer Letters 158, 2000, 179-184).

Therefore a need exists to find molecules that inhibit the activity of telomerase and interfere with the growth of many types of cancer.

The present invention fulfills such a need by providing a highly general method of treating many—if not most—malignancies, as demonstrated by the highly varied human tumor cell lines and tumors having telomerase activity.

Since the compounds of the present invention can be effective in providing treatments that discriminate between malignant and normal cells to a high degree, avoiding many of the deleterious side-effects present with most current chemotherapeutic regimes which rely on agents that kill dividing cells indiscriminately, they are also expected to exhibit greater safety and lack of toxic effects in comparison with traditional chemotherapeutic anticancer agents.

### SUMMARY OF THE INVENTION

The present invention relates to known and novel substituted benzopyranones active as telomerase inhibitors, to their use as therapeutic agents, in particular as antitumoral agents, to a process for their preparation and to pharmaceutical compositions comprising them.

These and other aspects of the invention are described in greater detail below.

### DETAILED DESCRIPTION OF THE INVENTION

It is an object of the present invention to provide a method for inhibiting telomerase enzyme, which comprises contacting said enzyme with an effective amount of a compound having the following formula (I)

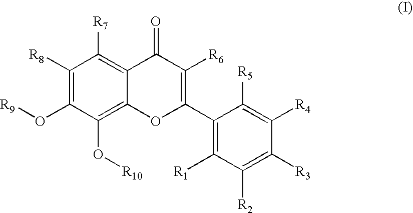

wherein

each of R₁, R₂ and R₅ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino, C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl;

each of R₃ and R₄ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino, C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl, or

R₃ and R₄, taken together, represent a 5 or 6 membered fused ring system having the following formula

—Y—(y)—X—(x)—Z—

wherein

(i) when X represents methylene, —(y)— represents a single bond, —(x)— represents a single bond, Y and Z both represent oxygen (O) or sulphur (S);

(ii) when X represents CR, —(y)— represents a double bond, —(x)— represents a single bond, Y represents N, Z represents NR′, oxygen (O) or sulphur (S), R represents hydrogen or C₁-C₆ alky optionally substituted with C₁-C₆ dialkylamino and R′ represents hydrogen, C₁-C₆ alkyl, or C₁-C₆ acyl;

(iii) when X represents C—OR, —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;

(iv) when X represents C═O, —(y)— represents a single bond, —(x)— represents a single bond, Y represents NR′, Z represents NR′, oxygen (O) or sulphur (S) and R′ represents hydrogen, C₁-C₆ alkyl, or C₁-C₆ acyl;

(v) when X represents C—SR, —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;

(vi) when X represents C═S, —(y)— represents a single bond, —(x)— represents a single bond, Y represents NR′, Z represents NR′, oxygen (O) or sulphur (S) and R′ represents hydrogen, C₁-C₆ alkyl or C₁-C₆ acyl;

(vii) when X represents N, —(y)— represents a double bond, —(x)— represents a single bond, Y represents N, Z represents NR′ and R′ represents hydrogen, C₁-C₆ alkyl or C₁-C₆ acyl;

(viii) when X represents O═C—C═O, —(y)— represents a single bond, —(x)— represents a single bond, Y and Z represent NR′ wherein R′ represents hydrogen, C₁-C₆ alkyl or C₁-C₆ acyl;

(ix) when X represents RO—C—C═O, —(y)— represents a double bond, —(x)— represents a single bond, Y represents N, Z represents NR′ and each of R and R′ represents, independently, hydrogen, C₁-C₆ alkyl or C₁-C₆ acyl;

(x) when X represents RO—C—C—OR, —(y)— represents a double bond, —(x)— represents a double bond, Y and Z are N, where R represents hydrogen, C₁-C₆ alkyl or C₁-C₆ acyl;

(xi) when X represents CH₂—CO, —(y)— represents a single bond, —(x)— represents a single bond, Y represents NR, Z represents NR′ and each of R and R′ represents, independently, hydrogen, C₁-C₆ alkyl or C₁-C₆ acyl;

(xii) when X represents CH₂—C—OR′, —(y)— represents a single bond, —(x)— represents a double bond, Y represents NR, Z represents N and each of R and R′ represents, independently, hydrogen, C₁-C₆ alkyl or C₁-C₆ acyl;

(xiii) when X represents CO—CH₂, —(y)— represents a single bond, —(x)— represents a single bond, Y represents NR′, Z represents NR and each of R and R′ represents, independently, hydrogen, C₁-C₆ alkyl or C₁-C₆ acyl; or

(xiv) when X represents R′O—C—CH₂, —(y)— represents a double bond, —(x)— represents a single bond, Y represents N, Z represents NR and each of R and R′ represents, independently, hydrogen, C₁-C₆ alkyl or C₁-C₆ acyl;

R₆ represents hydrogen, halogen, cyano, NRₐR_{b} in which each of Rₐ and R_{b} represents, independently, hydrogen, C₁-C₆ alkyl, C₁-C₄ acyl, aroyl, C₁-C₆ alkylsulfonyl or arylsulfonyl;

each of R₇ and R₈ represents, independently, hydrogen, halogen, cyano, C₁-C₆ alkoxy, C₁-C₆ acyloxy, aroyloxy, hydroxy, C₁-C₆ trialkylsilyloxy, aryl C₁-C₆ dialkylsilyloxy, alkyl C₁-C₆ diarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl;

each of R₉ and R₁₀ represents, independently, hydrogen; C₁-C₆ alkyl unsubstituted or substituted by aryl; C₁-C₆ acyl; aroyl; C₁-C₆ trialkylsilyl; aryl C₁-C₆ dialkylsilyl; C₁-C₆ alkyldiarylsilyl; triarylsilyl;

C₁-C₆ alkoxycarbonyl; or R₉ and R₁₀, taken together, represent methylene or carbonyl;

or a pharmaceutically acceptable salts thereof.

It is a further object of the present invention to provide a method for treating a telomerase-modulated disease, which comprises administering to a mammal a therapeutic effective amount of a compound having the above formula (I) or a pharmaceutically acceptable salt thereof.

It is a still further object of the present invention a method for treating a cancer disease related to a deranged cancer cell growth mediated by telomerase enzyme activity, which comprises administering to a mammal a therapeutic effective amount of a compound having the above formula (I) or a pharmaceutically acceptable salt thereof.

It is another object of the present invention to provide a method for treating a cancer, which comprises administering to a mammal a therapeutic effective amount of a compound having the above formula (I) or a pharmaceutically acceptable salt thereof.

According to still another aspect of the invention, a method is provided which involves the use of a compound having the above formula (I) in the preparation of a medicament. In particular embodiments, the medicament is for treating a proliferative disorder (e.g. a cancer). The present invention therefore also provides a compound having the above formula (I) for use in the preparation of a medicament having anticancer activity.

The present invention also comprises in its scope a pharmaceutical formulation for treating a telomerase-modulated disease, which comprises a compound having the above formula (I) or a pharmaceutically acceptable salt thereof and a pharmaceutically acceptable excipient.

The present invention also comprises in its scope a pharmaceutical formulation for treating a cancer disease related to a deranged cancer cell growth mediated by telomerase enzyme activity, which comprises a compound having the above formula (I) or a pharmaceutically acceptable salt thereof and a pharmaceutically acceptable excipient.

The present invention also comprises in its scope a pharmaceutical formulation for treating a cancer, which comprises a compound having the above formula (I) or a pharmaceutically acceptable salt thereof and a pharmaceutically acceptable excipient.

Some compounds of the aforementioned substituted benzopyranones of formula (I) and the pharmaceutically acceptable salt thereof are novel compounds and, as such, they represent a still another object of the present invention. Thus, the present invention also provides:

compounds of formula (Ia) having the same graphic structure of formula (I) as defined above wherein:

R₆ represents halogen, cyano, NRₐR_{b} in which each of Rₐ and R_{b} represents, independently, hydrogen, C₁-C₆ alkyl, C₁-C₄ acyl, aroyl, C₁-C₆ alkylsulfonyl or arylsulfonyl;

each of R₃, R₄ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl, or

R₃ and R₄, taken together, represent a 5 or 6 membered fused ring system having the following formula

—Y—(y)—X—(x)—Z—

wherein

(i) when X represents methylene, —(y)— represents a single bond, —(x)— represents a single bond, Y and Z both represent oxygen (O) or sulphur (S);

(ii) when X represents CR, —(y)— represents a double bond, —(x)— represents a single bond, Y represents N, Z represents NR′, oxygen (O) or sulphur (S), R represents hydrogen or C₁-C₆ alky optionally substituted with C₁-C₆ dialkylamino and R′ represents hydrogen, C₁-C₆ alkyl, or C₁-C₆ acyl;

(iii) when X represents C—OR , —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;

(iv) when X represents C═O, —(y)— represents a single bond, —(x)— represents a single bond, Y represents NR′, Z represents NR′, oxygen (O) or sulphur (S) and R′ represents hydrogen, C₁-C₆ alkyl, or C₁-C₆ acyl;

(v) when X represents C—SR, —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;

(vi) when X represents C═S, —(y)— represents a single bond, —(x)— represents a single bond, Y represents NR′, Z represents NR′, oxygen (O) or sulphur (S) and R′ represents hydrogen, C₁-C₆ alkyl or C₁-C₆ acyl;

(vii) when X represents N, —(y)— represents a double bond, —(x)— represents a single bond, Y represents N, Z represents NR′ and R′ represents hydrogen, C₁-C₆ alkyl or C₁-C₆ acyl;

(viii) when X represents O═C—C═O, —(y)— represents a single bond, —(x)— represents a single bond, Y and Z represent NR′ wherein R′ represents hydrogen, C₁-C₆ alkyl or C₁-C₆ acyl;

(ix) when X represents RO—C—C═O, —(y)— represents a double bond, —(x)— represents a single bond, Y represents N, Z represents NR′ and each of R and R′ represents, independently, hydrogen, C₁-C₆ alkyl or C₁-C₆ acyl;

(x) when X represents RO—C—C—OR, —(y)— represents a double bond, —(x)— represents a double bond, Y and Z are N, where R represents hydrogen, C₁-C₆ alkyl or C₁-C₆ acyl;

(xi) when X represents CH₂—CO, —(y)— represents a single bond, —(x)— represents a single bond, Y represents NR, Z represents NR′ and each of R and R′ represents, independently, hydrogen, C₁-C₆ alkyl or C₁-C₆ acyl;

(xii) when X represents CH₂—C—OR′, —(y)— represents a single bond, —(x)— represents a double bond, Y represents NR, Z represents N and each of R and R′ represents, independently, hydrogen, C₁-C₆ alkyl or C₁-C₆ acyl;

(xiii) when X represents CO—CH₂, —(y)— represents a single bond, —(x)— represents a single bond, Y represents NR′, Z represents NR and each of R and R′ represents, independently, hydrogen, C₁-C₆ alkyl or C₁-C₆ acyl; or

(xiv) when X represents R′O—C—CH₂, —(y)— represents a double bond, —(x)— represents a single bond, Y represents N, Z represents NR and each of R and R′ represents, independently, hydrogen, C₁-C₆ alkyl or C₁-C₆ acyl;

each of R₁, R₂ and R₅ represents, independently, hydrogen, halogen, cyano, hydroxy, C₁-C₆ alkoxy, C₁-C₆ acyloxy, aroyloxy, C₁-C₆ trialkylsilyloxy, ary C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl, or arylaminosulfonyl;

each of R₇ and R₈ represents, independently, hydrogen, halogen, cyano, C₁-C₆ alkoxycarbonyl, carboxyl, nitro, amino, C₁-C₆ monoalkylamino C₁-C₆ dialkylamino, C₁-C₆ trialkylammnonium halides, C₁-C₄ acylamino, aroylamino, C₁-C₆ alkylsulfonylamino, arylsulfonylamino, C₁-C₆ alkylaminosulfony or arylaminosulfonyl;

each of R₉ and R₁₀ represents, independently, hydrogen; C₁-C₆ alkyl unsubstituted or substituted by aryl; C₁-C₆ acyl; aroyl; C₁-C₆ trialkylsilyl; aryl C₁-C₆ dialkylsilyl; C₁-C₆ alkyldiarylsilyl; triarylsilyl; C₁-C₆, alkoxycarbonyl; or R₉ and R₁₀, taken together, represent methylene or carbonyl;

or a pharmaceutically acceptable salt thereof;

compounds of formula (Ib) having the same graphic structure of formula (I) as defined above wherein:

R₆ is hydrogen;

R₃ and R₄, taken together, represent a 5 or 6 membered fused ring system having the following formula

—Y—(y)—X—(x)—Z—

wherein

(ii) when X represents CR, —(y)— represents a double bond, —(x)— represents a single bond, Y represents N, Z represents NR′, oxygen (O) or sulphur (S), R represents hydrogen or C₁-C₆ alky optionally substituted with C₁-C₆ dialkylamino and R′ represents hydrogen, C₁-C₆ alkyl, or C₁-C₆ acyl;

(iii) when X represents C—OR , —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;

(iv) when X represents CO═O, —(y)— represents a single bond, —(x)— represents a single bond, Y represents NR′, Z represents NR′, oxygen (O) or sulphur (S) and R′ represents hydrogen, C₁-C₆ alkyl, or C₁-C₆ acyl;

(v) when X represents C—SR, —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;

(vi) when X represents C═S, —(y)— represents a single bond, —(x)— represents a single bond, Y represents NR′, Z represents NR′, oxygen (O) or sulphur (S) and R′ represents hydrogen, C₁-C₆ alkyl or C₁-C₆ acyl;

(vii) when X represents N, —(y)— represents a double bond, —(x)— represents a single bond, Y represents N, Z represents NR′ and R′ represents hydrogen, C₁-C₆ alkyl or C₁-C₆ acyl;

(viii) when X represents O═C—C═O, —(y)— represents a single bond, —(x)— represents a single bond, Y and Z represent NR′ wherein R′ represents hydrogen, C₁-C₆ alkyl or C₁-C₆ acyl;

(ix) when X represents RO—C—C═O, —(y)— represents a double bond, —(x)— represents a single bond, Y represents N, Z represents NR′ and each of R and R′ represents, independently, hydrogen, C₁-C₆ alkyl or C₁-C₆ acyl;

(x) when X represents RO—C—C—OR, —(y)— represents a double bond, —(x)— represents a double bond, Y and Z are N, where R represents hydrogen, C₁-C₆ alkyl or C₁-C₆ acyl;

(xi) when X represents CH₂—CO, —(y)— represents a single bond, —(x)— represents a single bond, Y represents NR, Z represents NR′ and each of R and R′ represents, independently, hydrogen, C₁-C₆ alkyl or C₁-C₆ acyl;

(xii) when X represents CH₂—C—OR′, —(y)— represents a single bond, —(x)— represents a double bond, Y represents NR, Z represents N and each of R and R′ represents, independently, hydrogen, C₁-C₆ alkyl or C₁-C₆ acyl;

(xiii) when X represents CO—CH₂, —(y)— represents a single bond, —(x)— represents a single bond, Y represents NR′, Z represents NR and each of R and R′ represents, independently, hydrogen, C₁-C₆ alkyl or C₁-C₆ acyl; or

(xiv) when X represents R′O—C—CH₂, —(y)— represents a double bond, —(x)— represents a single bond, Y represents N, Z represents NR and each of R and R′ represents, independently, hydrogen, C₁-C₆ alkyl or C₁-C₆ acyl;

each of R₁, R₂ and R₅ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl;

each of R₇ and R₈ represents, independently, hydrogen, halogen, cyano, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl, or arylaminosulfonyl;

each of R₉ and R₁₀ represents, independently, C₁-C₆ acyl; aroyl, C₁-C₆ trialkylsilyl; aryl C₁-C₆ dialkylsilyl; C₁-C₆ alkyldiarylsilyl; triarylsilyl; C₁-C₆ alkoxycarbonyl; or R₉ and R₁₀, taken together, represent methylene or carbonyl;

or a pharmaceutically acceptable salt thereof; and

compounds of formula (Ic) having the same graphic structure of formula (I) as defined above wherein:

R₆ is hydrogen;

each of R₃ and R₄ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ di alkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino arylsulfonylamino, C₁-C₆ alkylaminosulfonyl, and arylaminosulfonyl;

each of R₁, R₂ and R₅ represents, independently, hydrogen, halogen, cyano, hydroxy, C₁-C₆ alkoxy, C₁-C₆ acyloxy, aroyloxy, C₁-C₆ trialkylsilyloxy, aryldi C₁-C₆ alkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆alkylsulfonylamino, arylsulfonylamino, C₁-C₆ alkylaminosulfonyl or arylaminosulfonyl;

each of R₇ and R₈ represents, independently, halogen, cyano, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl, or arylaminosulfonyl, provided that R₇ and R₈ are not contemporarily hydrogen;

each of R₉ and R₁₀ represents, independently, hydrogen; C₁-C₆ alkyl unsubstituted or substituted by aryl; C₁-C₆ acyl; aroyl; C₁-C₆ trialkylsilyl; aryl C₁-C₆ dialkylsilyl; C₁-C₆ alkyldiarylsilyl; triarylsilyl; or C₁-C₆ alkoxycarbonyl; or R₉ and R₁₀, taken together, represent methylene or a carbonyl;

or a pharmaceutically acceptable salt thereof.

The compounds of formula (Ia), (Ib) and (Ic) represent selected classes of compounds of formula (I) and are thus also effective as telomerase inhibitors and active in the treatment of all the diseases for which the compounds of formula (I) have been indicated as therapeutic agents. A particular class of compounds of formula (Ia) according to the invention are compounds of formula (Ia) wherein:

R₆ is as defined in formula (Ia) above;

each of R₃ and R₄ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl, or

R₃ and R₄, taken together, represent a 5 or 6 membered fused ring system having the following formula

—Y—(y)—X—(x)—Z—

wherein

(i) when X represents methylene, —(y)— represents a single bond, —(x)— represents a single bond, Y and Z both represent oxygen (O) or sulphur (S);

(iii)when X represents CR, —(y)— represents a double bond, —(x)— represents a single bond, Y represents N, Z represents NR′, oxygen (O) or sulphur (S), R represents hydrogen or C₁-C₆ alky optionally substituted with C₁-C₆ dialkylamino and R′ represents hydrogen or C₁-C₆ alkyl;

(iii) when X represents C—OR , —(y)— represents a double bond, —(x)— represents a single bond, Y represents N, Z represents NR′, oxygen (O) or sulphur (S) and each of R and R′ represents, independently, hydrogen or C₁-C₆ alkyl;

(iv) when X represents C═O, —(y)— represents a single bond, —(x)— represents a single bond, Y represents NR′, Z represents NR′, oxygen (O) or sulphur (S) and R′ represents hydrogen or C₁-C₆ alkyl;

(v) when X represents C—SR, —(y)— represents a double bond, —(x)— represents a single bond, Y represents N, Z represents NR′, oxygen (O) or sulphur (S) and each of R and R′ represents, independently, hydrogen or C₁-C₆ alkyl;

(vi) when X represents C═S, —(y)— represents a single bond, —(x)— represents a single bond, Y represents NR′, Z represents NR′, oxygen (O) or sulphur (S) and R′ represents hydrogen or C₁-C₆ alkyl;

(vii) when X represents N, —(y)— represents a double bond, —(x)— represents a single bond, Y represents N, Z represents NR′ and R′ represents hydrogen or C₁-C₆ alkyl;

(viii) when X represents O═C—C═O, —(y)— represents a single bond, —(x)— represents a single bond, Y and Z represent NR′ wherein R′ represents hydrogen or C₁-C₆ alkyl;

(ix) when X represents RO—C—C═O, —(y)— represents a double bond, —(x)— represents a single bond, Y represents N, Z represents NR′ and each of R and R′ represents, independently, hydrogen or C₁-C₆ alkyl;

(x) when X represents RO—C—C—OR, —(y)— represents a double bond, —(x)— represents a double bond, Y and Z represent N and R represents hydrogen or C₁-C₆ alkyl;

(xi) when X represents CH₂—CO, —(y)— represents a single bond, —(x)— represents a single bond, Y represents NR, Z represents NR′ and each of R and R′ represents, independently, hydrogen or C₁-C₆ alkyl;

(xii) when X represents CH₂—C—OR′, —(y)— represents a single bond, —(x)— represents a double bond, Y represents NR, Z represents N and each of R and R′ represents, independently, hydrogen or C₁-C₆ alkyl;

(xiii) when X represents CO—CH₂, —(y)— represents a single bond, —(x)— represents a single bond, Y represents NR′, Z represents NR and each of R and R′ represents, independently, hydrogen or C₁-C₆ alkyl; or

(xiv) when X represents R′O—C—CH₂, —(y)— represents a double bond, —(x)— represents a single bond, Y represents N, Z represents NR and each of R and R′ represents, independently, hydrogen or C₁-C₆ alkyl;

each of R₁, R₂ and R₅ represents, independently, hydrogen, halogen, hydroxy, C₁-C₆ alkoxy, , C₁-C₆ acyloxy or aroyloxy;

each of R₇ and R₈ represents, independently, hydrogen or halogen;

each of R₉ and R₁₀ represents, independently, hydrogen; C₁-C₆ alkyl unsubstituted or substituted by aryl; C₁-C₆ acyl; aroyl; or R₉ and R₁₀, taken together, represent methylene;

or a pharmaceutically acceptable salt thereof.

A particular class of compounds of formula (Ib) according to the invention are compounds of formula (Ib) wherein:

R₆ is as defined in formula (Ib) above;

R₃ and R₄, taken together, represent a 5 or 6 membered fused ring system having the following formula

—Y—(y)—X—(x)—Z—

wherein

(ii) when X represents CR, —(y)— represents a double bond, —(x)— represents a single bond, Y represents N, Z represents NR′, oxygen (O) or sulphur (S), R represents hydrogen or C₁-C₆ alky optionally substituted with C₁-C₆ dialkylamino and R′ represents hydrogen or C₁-C₆ alkyl;

(iii) when X represents C—OR , —(y)— represents a double bond, —(x)— represents a single bond, Y represents N, Z represents NR′, oxygen (O) or sulphur (S) and each of R and R′ represents, independently, hydrogen or C₁-C₆ alkyl;

(iv) when X represents C═O, —(y)— represents a single bond, —(x)— represents a single bond, Y represents NR′, Z represents NR′, oxygen (O) or sulphur (S) and R′ represents hydrogen or C₁-C₆ alkyl;

(v) when X represents C—SR, —(y)— represents a double bond, —(x)— represents a single bond, Y represents N, Z represents NR′, oxygen (O) or sulphur (S) and each of R and R′ represents, independently, hydrogen or C₁-C₆ alkyl;

(vi) when X represents C═S, —(y)— represents a single bond, —(x)— represents a single bond, Y represents NR′, Z represents NR′, oxygen (O) or sulphur (S) and R′ represents hydrogen or C₁-C₆ alkyl;

(vii) when X represents N, —(y)— represents a double bond, —(x)— represents a single bond, Y represents N, Z represents NR′ and R′ represents hydrogen or C₁-C₆ alkyl;

(viii) when X represents O═C—C═O, —(y)— represents a single bond, —(x)— represents a single bond, Y and Z represent NR′ wherein R′ represents hydrogen or C₁-C₆ alkyl;

(ix) when X represents RO—C—C═O, —(y)— represents a double bond, —(x)— represents a single bond, Y represents N, Z represents NR′ and each of R and R′ represents, independently, hydrogen or C₁-C₆ alkyl;

(x) when X represents RO—C—C—OR, —(y)— represents a double bond, —(x)— represents a double bond, Y and Z represent N and R represents hydrogen or C₁-C₆ alkyl;

(xi) when X represents CH₂—CO, —(y)— represents a single bond, —(x)— represents a single bond, Y represents NR, Z represents NR′ and each of R and R′ represents, independently, hydrogen or C₁-C₆ alkyl;

(xii) when X represents CH₂—C—OR′, —(y)— represents a single bond, —(x)— represents a double bond, Y represents NR, Z represents N and each of R and R′ represents, independently, hydrogen or C₁-C₆ alkyl;

(xiii) when X represents CO—CH₂, —(y)— represents a single bond, —(x)— represents a single bond, Y represents NR′, Z represents NR and each of R and R′ represents, independently, hydrogen or C₁-C₆ alkyl; or

(xiv) when X represents R′O—C—CH₂, —(y)— represents a double bond, —(x)— represents a single bond, Y represents N, Z represents NR and each of R and R′ represents, independently, hydrogen or C₁-C₆ alkyl;

each of R₁, R₂ and R₅ represents, independently, hydrogen, halogen, hydroxy, C₁-C₆ alkoxy, C₁-C₆ acyloxy or aroyloxy;

each of R₇ and R₈ represents, independently, hydrogen or halogen;

each of R₉ and R₁₀ represents, independently, hydrogen; C₁-C₆ alkyl unsubstituted or substituted by aryl; C₁-C₆ acyl; aroyl; or R₉ and R₁₀, taken together, represent methylene;

or a pharmaceutically acceptable salt thereof.

A particular class of compounds of formula (Ic) according to the invention are compounds of formula (Ic) wherein:

R₆ is as defined in formula (Ic) above;

each of R₁, R₂ and R₅ represents, independently, hydrogen, halogen, hydroxy, C₁-C₆ alkoxy, C₁-C₆ acyloxy or aroyloxy;

each of R₇ and R₈ represents, independently, halogen, cyano, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl, or arylaminosulfonyl, provided that R₇ and R₈ are not contemporarily hydrogen;

each of R₉ and R₁₀ represents, independently, hydrogen; C₁-C₆ alkyl unsubstituted or substituted by aryl; C₁-C₆ acyl; aroyl; or R₉ and R₁₀, taken together, represent methylene,

or a pharmaceutically acceptable salt thereof.

Pharmaceutically acceptable salts of the compounds of formula (I), (Ia), (Ib) and (Ic) are their salts with pharmaceutically acceptable either inorganic or organic acids such as, for instance, hydrochloric, hydrobromic, sulfuric, nitric, acetic, propionic, succinic, malonic, citric, tartaric, methanesulfonic and p-toluensulfonic acid, and their salts with pharmaceutically accceptable either inorganic or organic bases such as, for instance, hydroxides of alkali metals, for example, sodium or potassium, or alkaline earth metals such as, for instance, calcium, magnesium, zinc or aluminium, and organic bases, such as, for instance, aliphatic amines such as, for instance, methyl amine, diethylamine, trimethylamine, ethylamine or heterocyclic amines such as, for instance, piperidine. Such salts can be formed as known to those skilled in the art.

By the term “halogen” as used herein, is meant chlorine, bromine, iodine or fluorine.

By the term “alkyl”, as used herein, either alone or within other terms, is meant an acyclic alkyl radical; the alkyl groups may be branched or straight chain groups.

By the term “alkoxy” as used herein, is meant O-alkyl groups wherein the term “alkyl”, is as defined above.

By the term “acyl” as used herein, either alone or within other terms, is meant alkyl groups as defined above attached to a carbonyl group, i.e. alkyl-C═O groups, for instance, formyl, acetyl, propanoyl, butanoyl and pentanoyl.

C₁-C₆ alkyl is, preferably, C₁-C₄ alkyl, in particular methyl or ethyl.

C₁-C₆ acyl is, preferably, C₁-C₄ acyl, in particular acetyl or propanoyl.

C₁-C₆ alkoxy is, preferably, C₁-C₄ alkoxy, typically methoxy, ethoxy, propoxy or butoxy.

C₁-C₆ acyloxy is, preferably, C₁-C₄ acyloxy, preferably acetyloxy or propionyloxy.

C₁-C₄ acylamino is, preferably, acetylamino or propionylamino.

C₁-C₆ alkoxycarbonyl group is, preferably, a C₁-C₄ alkoxycarbonyl group typically a C₁-C₂ one.

By the term “aryl” as used herein, is meant an aromatic system having 20 or fewer carbon atoms, which may be a single ring or multiple aromatic rings fused or linked together as such that at least one part of the fused or linked rings forms the conjugated aromatic system. The aryl groups as defined immediately above, include but not limited to phenyl, naphthyl, biphenyl and anthracenyl. The aryl groups as just defined above, may be optionally substituted by from one to four substituents from the group including halogen, cyano, hydroxy, nitro, amino, C₁-C₆ monoalkylamino, C₁-C₆ dialkylamino, C₁-C₆ alkyl, cycloalkyl, C₁-C₆ alkylaryl, alkenyl, alkynyl, aryl, 5-10 membered heterocyclyl, alkoxy, aryloxy, C₁-C₆ alkylthio, arylthio, C₁-C₆ alkylsulfonyl, arylsulfonyl, C₁-C₆ acyl, aroyl, C₁-C₆ acyloxy, C₁-C₄ acylamino, C₁-C₆ alkoxycarbonyl, aryloxycarbonyl, carboxyl, C₁-C₆ alkylsulfonylamino, arylsulfonylamino, C₁-C₆ alkylaminosulfonyl and arylaminosulfonyl.

By the term “cycloalkyl” as used herein, is meant a C₁-C₁₀ all-carbon monocyclic or fused ring, including, e.g., cyclopropane, cyclobutane, cyclopentane, cyclohexane and cycloheptane.

By the term “alkenyl” as used herein, is meant an alkyl group, as defined herein, consisting of at least two carbon atoms and at least one carbon-carbon double bond.

By the term “alkynyl” as used herein, is meant an alkyl group, as defined herein, consisting of at least two carbon atoms and at least one carbon-carbon triple bond.

By the term “5-10 membered heterocyclyl” as used herein, is meant aromatic and non-aromatic heterocyclic groups containing one or more heteroatoms each selected from O, S and N, wherein each heterocyclic group has from 5-10 atoms in its ring system. Examples of non-aromatic heterocyclic groups are pyrrolidinyl, piperidino, morpholino, thiomorpholino and piperazinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl and thiazolyl. Heterocyclic groups having a fused benzene ring include benzimidazolyl.

By the term “aroyl” as used herein, is meant aryl groups, as defined above, attached to a carbonyl group, i.e. aryl-C═O, for instance benzoyl and toluoyl.

The term “sulfonyl”, used alone or linked to other terms such as, for instance, alkylsulfonyl or arylsulfonyl, denotes respectively divalent radicals-SO₂—. The term “alkylsulfonyl”, embraces alkyl radicals attached to a sulfonyl radical, where alkyl is as defined above. The term “arylsulfonyl”, embraces aryl radicals attached to a sulfonyl radical, where aryl is as defined above.

The terms “malignant neoplasm”, “cancer”, “tumor” and “solid tumor cancer” are used interchangeably herein to refer to the condition well known to those skilled in the art as the life-threatening disease commonly referred to simply as “cancer”.

The term “cancer” as used herein, is meant a disease characterized by excessive, uncontrolled growth of abnormal cells, which invades and destroys other tissues and includes all human cancers such as carcinomas, sarcomas, leukemias and lymphomas. For example, the term “cancer” comprises prostate, breast, lung, colorectal, bladder, uterine, skin, kidney, pancreatic, ovarian, liver and stomach cancer.

By the term “chemotherapeutic agent” as used herein, is meant a chemical substance or drug used to treat a disease; the term is most often applied to such substances or drugs which are used primarily for the treatment of cancer.

By the term “treating” as used herein, is meant reversing, alleviating, ameliorating, limiting, inhibiting the progress of, or preventing the disorder or condition to which such term applies, or one or more symptoms of such disorder or condition. The term “treatment” as used herein, refers to the act of treating as “treating” is defined immediately above.

By the term “method” as used herein, is meant manners, means, techniques and procedures for accomplishing a given task including, but not limited to, those manners, means, techniques and procedures either known to, or readily developed from known manners, means, techniques and procedures by, practitioners of the chemical, pharmacological, biological, biochemical and medical arts.

By the term “administered” or “administering” as used herein, is meant standard delivery methods, e.g, parenteral administration, including continuous infusion and intravenous, intramuscular and subcutaneous injections, and oral administration.

The term “modulated” as used herein, includes governed, controlled, provoked, modulated and induced.

By the term “mammal” as used herein, is meant any of a class of warm-blooded higher vertebrates, that nourish their young with milk secreted by mammary glands, have the skin usually more or less covered with hair, and includes humans.

By the term “physiologically acceptable carrier” as used herein, is meant a carrier or diluent that does not cause significant irritation to an organism and does not abrogate the biological activity and properties of the administered compound.

By the term “excipient” as used herein, is meant an inert substance added to a pharmaceutical composition to further facilitate administration of a compound.

By the term “disease” as used herein, is meant a kind or instance of impairment of a living being that interferes with normal bodily function.

The compounds of this invention may contain an asymmetric carbon atom and some of the compounds of this invention may contain one or more asymmetric centers and may thus give rise to optical isomers and diastereomers. While shown without respect to stereochemistry in formula (I), (Ia), (Ib) and (Ic), the present invention includes such optical isomers and diastereomers; as well as the racemic and resolved, enantiomerically pure R and S stereoisomers; as well as other mixtures of the R and S stereoisomers and pharmaceutically acceptable salts thereof.

Some of the compounds described herein may contain one or more ketonic or aldehydic carbonyl groups or combinations thereof alone or as part of a heterocyclic ring system. Such carbonyl groups may exist in part or principally in the “keto” form and in part or principally as one or more “enol” forms of each aldehyde and ketone group present. Compounds of the present invention having aldehydic or ketonic carbonyl groups are meant to include both “keto” and “enol” tautomeric forms. Some of the compounds described herein may contain one or more imine or enamine groups or combinations thereof. Such groups may exist in part or principally in the “imine” form and in part or principally as one or more “enamine” forms of each group present. Compounds of the present invention having said imine or enamine groups are meant to include both “imine” and “enamine” tautomeric forms.

It is therefore understood that the present invention includes in its scope all the possible tautomeric forms of the compounds of formula (I), (Ia), (Ib) and (Ic).

The present invention also includes within its scope pharmaceutically acceptable bio-precursors (otherwise known as pro-drugs) of the compounds of formula (I), (Ia), (Ib) and (Ic) above, i.e. compounds which have a different formula (I), (Ia), (Ib) and (Ic), but which nevertheless upon administration to a human being are converted directly or indirectly in vivo into a compound of formula (I), (Ia), (Ib) or (Ic).

Examples of specific compounds of the invention include:

7,8-dihydroxy-2-phenyl-4H-chromen-4-one (compound 1);

7,8-dihydroxy-2-(4-hydroxyphenyl)-4H-chromen-4-one (compound 2);

2-(3,4-dihydroxyphenyl)-7,8-dihydroxy-4H-chromen-4-one (compound 3);

7,8-dihydroxy-2-(2-hydroxyphenyl)-4H-chromen-4-one (compound 4);

7,8-dihydroxy-2-(3-hydroxyphenyl)-4H-chromen-4-one (compound 5);

7,8-dihydroxy-2-(4-methoxyphenyl)-4H-chromen-4-one (compound 6);

7,8-dihydroxy-2-(3,4-dimethoxyphenyl)-4H-chromen-4-one (compound 7);

7,8-dihydroxy-2-(3,4-methylenedioxyphenyl)-4H-chromen-4-one (compound 8);

7,8-dihydroxy-2-(2,3-dihydroxyphenyl)-4H-chromen-4-one (compound 9);

7,8-dihydroxy-2-(3,4,5-trihydroxyphenyl)-4H-chromen-4-one (compound 10);

7,8-dihydroxy-2-(2,3,4-trihydroxyphenyl)-4H-chromen-4-one (compound 11);

7,8-dihydroxy-2-(3,5-dimethoxy-4-hydroxyphenyl)-4H-chromen-4-one (compound 12);

7,8-dihydroxy-2-(3,4,5-trimethoxyphenyl)-4H-chromen-4-one (compound 13);

2-(3,4-diacetoxyphenyl)-7,8-diacetoxy-4H-chromen-4-one (compound 14);

5,7,8-trihydroxy-2-(3,4,5-trimethoxyphenyl)-4H-chromen-4-one (compound 15);

5,7,8-trihydroxy-2-(3,4,5-trihydroxyphenyl)-4H-chromen-4-one (compound 16);

7,8-dihydroxy-2-(4-nitrophenyl)-4H-chromen-4-one (compound 17);

7,8-dimethoxy-2-phenyl-4H-chromen-4-one (compound 18);

8-acetoxy-7-hydroxy-2-phenyl-4H-chromen-4-one (compound 19);

8-hydroxy-7-methoxy-2-phenyl-4H-chromen-4-one (compound 20);

7,8-dimethoxy-2-(3-nitrophenyl)-4H-chromen-4-one (compound 21);

7-hydroxy-8-methoxy-2-(4-methoxyphenyl)-4H-chromen-4-one (compound 22);

7,8-dimethoxy-2-(4-methoxyphenyl)-4H-chromen-4-one (compound 23);

7,8-dimethoxy-2-(3,4-dimethoxyphenyl)-4H-chromen-4-one (compound 24);

7,8-dimethoxy-2-(2-hydroxyphenyl)-4H-chromen-4-one (compound 25);

7,8-dimethoxy-2-(2-methoxyphenyl)-4H-chromen-4-one (compound 26);

7,8-dimethoxy-2-(3-hydroxyphenyl)-4H-chromen-4-one (compound 27);

7,8-dimethoxy-2-(2,3-dimethoxyphenyl)-4H-chromen-4-one (compound 28);

7,8-dimethoxy-2-(2,6-dihydroxyphenyl)-4H-chromen-4-one (compound 29);

7,8-dimethoxy-2-(4,5-dimethoxy-3-hydroxyphenyl)-4H-chromen-4-one (compound 30);

7,8-dimethoxy-2-(4-acetoxy-3,5-dimethoxyphenyl)-4H-chromen-4-one (compound 31);

7,8-diacetoxy-2-phenyl-4H-chromen-4-one (compound 32);

7,8-diacetoxy-2-(4-methoxyphenyl)-4H-chromen-4-one (compound 33);

7,8-diacetoxy-2-(2,3-diacetoxyphenyl)-4H-chromen-4-one (compound 34);

8-hydroxy-7-methoxy-2-(4,5-dimethoxy-3-hydroxyphenyl)-4H-chromen-4-one (compound 35);

2-(3,4-dihydroxyphenyl)-7,8-dihydroxy-6-methoxy-4H-chromen-4-one (compound 36)

and, if the case, the pharmaceutically acceptable salts thereof.

In a further aspect, the present invention provides a method for inhibiting telomerase enzyme, which comprises contacting said enzyme with an effective amount of a compound selected from the group of compounds 1-36 as defined above or a pharmaceutically acceptable salt thereof.

In a still further aspect, the present invention relates to a method for treating a telomerase-modulated disease, which comprises administering to a mammal a therapeutic effective amount of a compound selected from the group of compounds 1-36 as defined above or a pharmaceutically acceptable salt thereof.

In an additional aspect, the present invention provides a method for treating a cancer disease related to a deranged cancer cell growth mediated by telomerase enzyme activity, which comprises administering to a mammal a therapeutic effective amount of a compound selected from the group of compounds 1-36 as defined above or a pharmaceutically acceptable salt thereof.

It is still another object of the present invention to provide a method for treating a cancer, which comprises administering to a mammal a therapeutic effective amount of a compound selected from the group of compounds 1-36 as defined above or a pharmaceutically acceptable salt thereof.

Additional examples of specific compounds of the invention include:

3-chloro-2-(3,4-dihydroxyphenyl)-7,8-dihydroxy-4H-chromen-4-one (compound 37);

3-chloro-2-(3,4-dimethoxyphenyl)-7,8-dimethoxy-4H-chromen-4-one (compound 38);

3-cyano-2-(3,4-dihydroxyphenyl)-7,8-dihydroxy-4H-chromen-4-one (compound 39);

3-cyano-2-(3,4-dimethoxyphenyl)-7,8-dimethoxy-4H-chromen-4-one (compound 40);

3-fluoro-2-(3,4-dihydroxyphenyl)-7,8-dihydroxy-4H-chromen-4-one (compound 41);

2-(3,4-dimethoxyphenyl)-3-fluoro-7,8-dimethoxy-4H-chromen-4-one (compound 42);

2-(4-fluorophenyl)-7,8-dihydroxy-4H-chromen-4-one(compound 43);

2-(3-fluorophenyl)-7,8-dihydroxy-4H-chromen-4-one(compound 44);

2-(3-chlorophenyl)-7,8-dihydroxy-4H-chromen-4-one (compound 45);

2-(3,4-dichlorophenyl)-7,8-dihydroxy-4H-chromen-4-one (compound 46);

5-chloro-2-(3,4-dihydroxyphenyl)-7,8-dihydroxy-4H-chromen-4-one (compound 47);

5-chloro-2-(3,4-dimethoxyphenyl)-7,8-dimethoxy-4H-chromen-4-one (compound 48);

6-chloro-2-(3,4-dihydroxyphenyl)-7,8-dihydroxy-4H-chromen-4-one (compound 49);

6-chloro-2-(3,4-dimethoxyphenyl)-7,8-dimethoxy-4H-chromen-4-one (compound 50);

2-(3,4-diaminophenyl)-7,8-dimethoxy-4H-chromen-4-one (compound 51);

2-(3,4-diaminophenyl)-7,8-dihydroxy-4H-chromen-4-one (compound 52);

N-[4-(7,8-dimethoxy-4-oxo-4H-chromen-2-yl)-2-nitrophenyl]acetamide (compound 53);

2-(4-acetylamino-3-nitrophenyl)-7,8-dihydroxy-4H-chromen-4-one (compound 54);

2-(4-amino-3-nitrophenyl)-7,8-dimethoxy-4H-chromen-4-one (compound 55);

2-(4-amino-3-nitrophenyl)-7,8-dihydroxy-4H-chromen-4-one (compound 56);

2-(3,4-diacetylaminophenyl)-7,8-dihydroxy-4H-chromen-4-one (compound 57);

N-[2-(acetylamino)-4-(7,8-dimethoxy-4-oxo-4H-chromen-2-yl)phenyl]acetamide (compound 58);

2-(3,4-di-trifluoroacetylaminophenyl)-7,8-dihydroxy-4H-chromen-4-one (compound 59);

2-(3,4-di-trifluoroacetylaminophenyl)-7,8-dimethoxy-4H-chromen-4-one (compound 60);

2-(1H-benzimidazol-5-yl)-7,8-dimethoxy-4H-chromen-4-one (compound 61);

2-(1H-benzimidazol-5-yl)-7,8-dihydroxy-4H-chromen-4-one (compound 62);

2-(1H-benzimidazol-5-yl)-8-hydroxy-7-methoxy-4H-chromen-4-one (compound 63);

4-(7,8-dihydroxy-4-oxo-4H-chromen-2-yl)benzoic acid (compound 64);

2-(1H-1,2,3-benzotriazol-5-yl)-7,8-dimethoxy-4H-chromen-4-one (compound 65);

2-(1H-1,2,3-benzotriazol-5-yl)-7,8-dihydroxy-4H-chromen-4-one (compound 66);

5-(7,8-dimethoxy-4-oxo-4H-chromen-2-yl)-1,3-dihydro-2H-benzimidazol-2-one (compound 67);

5-(7,8-dihydroxy-4-oxo-4H-chromen-2-yl)-1,3-dihydro-2H-benzimidazol-2-one (compound 68);

7,8-dimethoxy-2-(2-sulfanyl-1H-benzimidazol-5-yl)-4H-chromen-4-one (compound 69);

7,8-dihydroxy-2-(2-sulfanyl-1H-benzimidazol-5-yl)-4H-chromen-4-one (compound 70);

2-(2-amino-1H-benzimidazol-5-yl)-7,8-dimethoxy-4H-chromen-4-one (compound 71);

2-(2-amino-1H-benzimidazol-5-yl)-7,8-dihydroxy-4H-chromen-4-one (compound 72);

2-(1,3-benzoxazol-5-yl)-7,8-dimethoxy-4H-chromen-4-one (compound 73);

2-(1,3-benzoxazol-5-yl)-7,8-dihydroxy-4H-chromen-4-one (compound 74);

2-(1,3-benzothiazol-5-yl)-7,8-dimethoxy-4H-chromen-4-one (compound 75);

2-(1,3-benzothiazol-5-yl)-7,8-dihydroxy-4H-chromen-4-one (compound 76);

2-(2,4-dihydroxyphenyl)-7,8-dihydroxy-4H-chromen-4-one (compound 77);

8-hydroxy-7-methoxy-2-(3,4-dihydroxyphenyl)-4H-chromen-4-one (compound 78);

7,8-dihydroxy-2-(3-hydroxy-4-methoxyphenyl)-4H-chromen-4-one (compound 79);

7,8-dimethoxy-2-(3,4-dihydroxyphenyl)-4H-chromen-4-one (compound 80);

6-(7,8-dimethoxy-4-oxo-4H-chromen-2-yl)-1,4-dihydro-2,3-quinoxalinedione (compound 81);

6-(7,8-dihydroxy-4-oxo-4H-chromen-2-yl)-1,4-dihydro-2,3-quinoxalinedione (compound 82);

6-(7,8-dimethoxy-4-oxo-4H-chromen-2-yl)-3,4-dihydro-2(1H)-quinoxalinone (compound 83);

6-(7,8-dihydroxy-4-oxo-4H-chromen-2-yl)-3,4-dihydro-2(1H)-quinoxalinone (compound 84);

7-(7,8-dimethoxy-4-oxo-4H-chromen-2-yl)-3,4-dihydro-2(1H)-quinoxalinone (compound 85);

7-(7,8-dihydroxy-4-oxo-4H-chromen-2-yl)-3,4-dihydro-2(1H)-quinoxalinone (compound 86);

7,8-dimethoxy-2-(3-hydroxy-4-methoxyphenyl)-4H-chromen-4-one (compound 87);

2-(2,4-dimethoxyphenyl)-7,8-dimethoxy-4H-chromen-4-one (compound 88);

4-(7,8-dihydroxy-4-oxo-4H-chromen-2-yl)benzonitrile (compound 89);

2-(3,4-diacetoxyphenyl)-7,8-dihydroxy-4H-chromen-4-one (compound 90);

7,8-dihydroxy-2-(4-hydroxy-3-methoxyphenyl)-4H-chromen-4-one (compound 91);

2-(3,4-dihydroxyphenyl)-7-hydroxy-8-methoxy-4H-chromen-4-one (compound 92);

7-hydroxy-2-(4-hydroxy-3-methoxyphenyl)-8-methoxy-4H-chromen-4-one (compound 93);

7-hydroxy-2-(3-hydroxy-4-methoxyphenyl)-8-methoxy-4H-chromen-4-one (compound 94);

8-hydroxy-2-(3-hydroxy-4-methoxyphenyl)-7-methoxy-4H-chromen-4-one (compound 95);

8-hydroxy-2-(4-hydroxy-3-methoxyphenyl)-7-methoxy-4H-chromen-4-one (compound 96);

2-(3-hydroxy-4-methoxyphenyl)-7,8-dimethoxy-4H-chromen-4-one (compound 97);

2-(3-fluoro-4-hydroxyphenyl)-7,8-dihydroxy-4H-chromen-4-one (compound 98);

2-(4-fluoro-3-hydroxyphenyl)-7,8-dihydroxy-4H-chromen-4-one (compound 99);

2-(3-hydroxy-4-methoxyphenyl)-7,8-dimethoxy-4H-chromen-4-one (compound 100);

7,8-dimethoxy-2-(2-methyl-1H-benzimidazol-5-yl)-4H-chromen-4-one (compound 101);

7,8-dihydroxy-2-(2-methyl-1H-benzimidazol-5-yl)-4H-chromen-4-one (compound 102);

2-{2-[3-(Dimethylamino)propyl]-1H-benzimidazol-5-yl}-7,8-dimethoxy-4H-chromen-4-one (compound 103) and, if the case, the pharmaceutically acceptable salts thereof.

An object of the present invention is also to provide a pharmaceutical composition, which comprises as an active principle a compound of formula (Ia), (Ib) or (Ic) as defined above or a pharmaceutically acceptable salt thereof and a pharmaceutically acceptable excipient.

Another object of the present invention is to provide a compound of formula (Ia), (Ib) or (Ic) as defined above or a pharmaceutically acceptable salt thereof for use as a medicament, in particular for the treatment of a telomerase-modulated disease, more in particular for the treatment of a cancer disease related to a deranged cancer cell growth mediated by telomerase enzyme activity, is encompassed by the scope of the present invention.

A Further object of the present invention is to provide a method for the preparation of compounds of formula (I) as defined above.

According to a preferred embodiment of the invention, a compound of formula (I) wherein R₁-R₅, R₇-R₁₀ are as defined in formula (I) above and R₆ is hydrogen, i.e. a compound of formula (III), can be prepared according to the process of Scheme 1, which comprises cyclization of a compound of formula (II) wherein R₁₁ is hydrogen or C₁-C₆ alkyl, preferably methyl and ethyl, and R₁-R₅, and R₇-R₁₀ are as defined above.

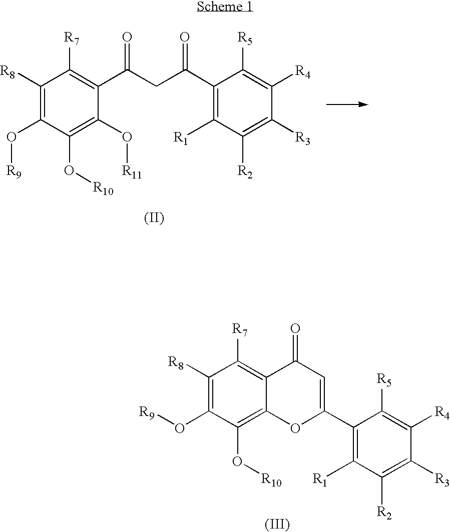

Typically a compound of formula (III) can be obtained by treating a compound of formula (II) under acidic conditions for 1 to 48 hrs, at temperature ranging from room temperature to refluxing conditions according to the solvent system used, according to methods of the literature (see, for example, The Flavonoids, “Synthesis of Flavonoids”, H. Wagner and L. Farkas, pg. 127-213, Ed. by J. B. Harborne, T. J. Mabry and H. Mabry, 1975 London). More typically a compound of formula (II) is heated in the presence of acetic acid, sulphuric acid, hydrochloric acid, hydrobromic acid, hydriodic acid or a combination of the aforementioned acids, at reflux for 1 to 24 hr. In particular, when R₁₁ is C₁-C₆ alkyl, for example methyl or ethyl, a compound of formula (II) is preferably treated with hydriodic acid in acetic acid, under reflux for 1 to 24 hr; under these conditions if alkoxy groups other than O—R₁₀ wherein R₁₀ is as defined above are present in the structure, they may be de-alkylated. In particular, when O—R₁₀ is methoxy, ethoxy or benzyloxy, the corresponding hydroxyl group may be present in the final product. In other cases when R₁₁ is hydrogen, a compound of formula (II) can be cyclized to a compound of formula (III) with anhydrous sodium acetate in glacial acetic acid, or sulphuric acid in glacial acetic acid.

In all cases where in the final product of formula (III) free hydroxy groups (OH) are present, for example when R₉ and R₁₀ are at the same time hydrogen and each of R₁-R₅ and R₇-R₈ represents hydroxy (OH), this product can be obtained by deprotection of a C₁-C₆ alkoxy (typically methoxy, ethoxy or benzyloxy) substituted precursor, or by deprotection of a silyloxy (typically trimethylsilyloxy, triisopropylsilyloxy, triethylsilyloxy, t-butyldimethylsilyloxy, or phenyldimethylsilyloxy) substituted precursor. For example, typical demethylation procedures require the use of an aqueous acid, such as, e.g., 57% HI or 48% HBr, eventually in the presence of glacial acetic acid, under temperatures ranging from room to refluxing temperature, for reaction time ranging from 1 to 72 hrs. In other cases the use of a Lewis acid such as, e.g., BBr₃, BCl₃, AlCl₃ or similar reagents is preferred, in the presence of a suitable organic solvent such as, e.g., methylene chloride, benzene or toluene, at temperatures ranging from −78° to 150° C., for 1 to 72 hrs. Most preferred among the previous Lewis acids is BBr₃. Typical debenzylation procedures consist of treatment of the benzyl derivatives under hydrogenolytic conditions, i.e. with hydrogen in the presence of a proper catalyst such as Pd on charcoal (like 5% or 10% Pd/C), or by using aqueous acidic conditions, for example the ones already reported for the demethylation process (i.e. 57% HI, 48% HBr with or without glacial acetic acid), or conc. HCl. Typical desilylation procedures involve the use of, for example, pyridinium hydrofluoric acid or tetrabutyl ammonium fluoride, in a suitable solvent such as, e.g., methylene chloride, THF, benzene or toluene, at temperatures ranging from −30 to 100° C. for 1 to 48 hrs. Alternatively, aqueous hydrochloric acid or hydrobromic acid in a suitable solvent such as, e.g., methanol, ethanol or similar might be used, at temperatures ranging from −20 to 150° C., for 1 to 48 hrs.

These deprotection procedures used to obtain flavones with free hydroxy groups can be considered of general utility and can be applied, unless otherwise specified, in almost all other cases of differently substituted flavones that will be described later on. Particular cases where the previous procedures cannot be applicable are specifically reported.

Particular class of compounds of the present invention characterized by specific substitution on the flavonic skeleton can be prepared according to the methods reported below.

For example, a class of compounds of formula (I) wherein R₆ represents chloro (Cl), R₁-R₅ and R₇-R₁₀ are as defined in formula (I) above, i.e. compounds of formula (IV)

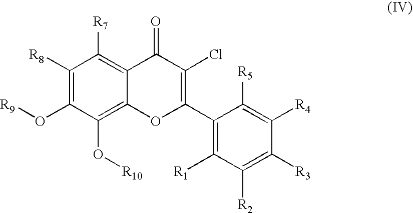

can be obtained by a process which comprises chlorination of a compound of formula (II) as defined above to achieve an intermediate of formula (V) wherein R₁-R₅ and R₇-R₁₀ are as defined in formula (II) above and ring closure to obtain a compound of formula (IV), according to the Scheme 2 below.

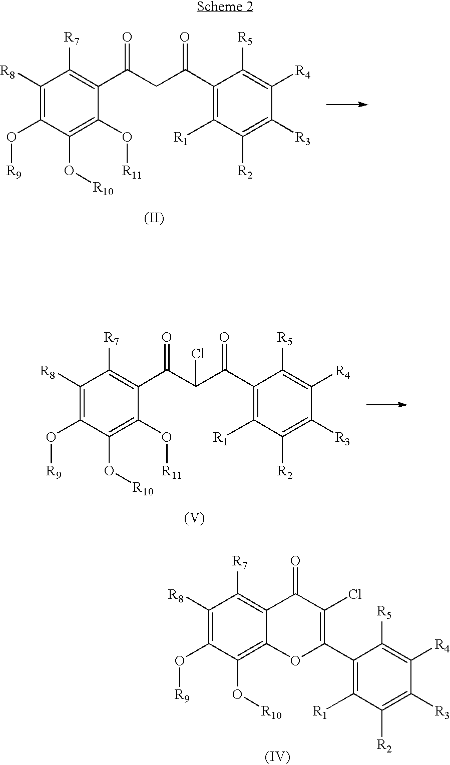

Under the chlorinating reaction conditions, intermediate of formula (V) cannot be isolated and it spontaneously cyclizes to afford compound of formula (IV). The chlorinating agent is, for example SO₂Cl₂, in a suitable organic solvent typically dioxane, under heating preferably refluxing conditions, for 1 to 12 hr, as described in the literature (see, for example, J. Med. Chem. 1991, 34, 736).

Another class of compounds of formula (I) of the present invention wherein R₆ represents fluoro (F), R₁-R₅ and R₇-R₁₀ are as defined in formula (I) above, i.e. compounds of formula (VI)

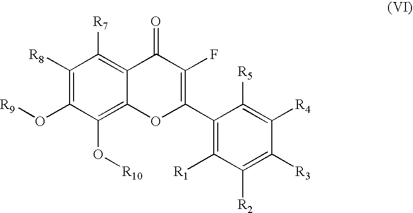

can be obtained by a process which comprises fluorination of a compound of formula (II) as defined above to achieve an intermediate of formula (VII) wherein R₁-R₅ and R₇-R₁₀ are as defined above and ring closure to obtain a compound of formula (VI), according to the Scheme 3 below.

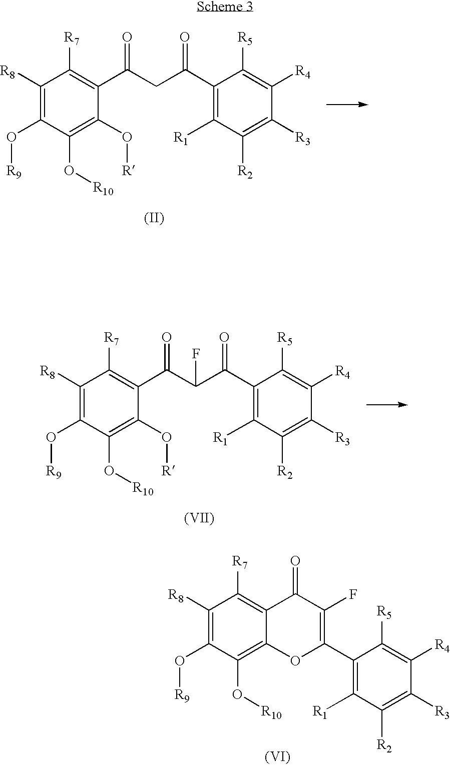

Intermediates of formula (VII) can be isolated and cyclized according to the same reaction conditions already described. A proper fluorinating reagent can be used, for example N-fluorobenzenesulfonimide or N-fluoro-o-benzenedisulfonimide, in an organic solvent such as, e.g. dichloromethane, as described in the literature (see, for example, Synlett 1991, 187; J. Org. Chem. 1995, 60, 4730). Alternatively a fluorinated tetrafluoroborate, for example 1-fluoro-4-hydroxy-1,4-diazoniabicyclo [2,2,2] octane bis (tetrafluoroborate) can be employed, tipically in an organic solvent such as, e.g. acetonitrile or methanol, under heating preferably refluxing conditions, for 1 to 12 hr, as described in the literature (see, for example, Tetr. Lett. 1996, 37, 3591).

A further class of compounds of formula (I) of the present invention wherein R₆ represents cyano (CN), R₁-R₅ and R₇-R₁₀ are as defined above, i.e. compounds of formula (VIII)

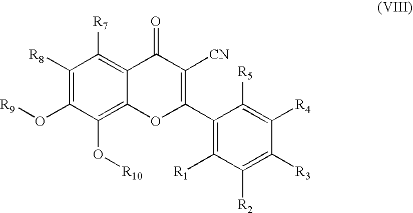

can be obtained by a process which comprises exchanging the chloro substituent in a compound of formula (IV) as defined above with a cyano group, according to the Scheme 4 below.

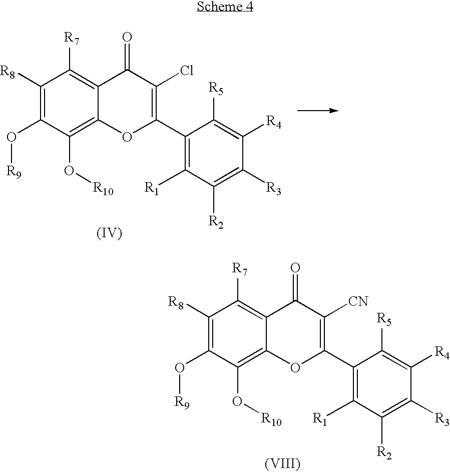

Typical exchange reaction conditions consist of treating a compound of formula (IV) with metal cyanide, like copper (rameous) cyanide (CuCN), in a suitable organic solvent comprising, e.g., DMF, DMSO, N,N-dimethyl acetamide or N-methyl pyrrolidone, under room to refluxing temperature conditions typically ranging from 100 to 220° C. for 2 to 48 hr, according to the literature (see, for example, J. Het. Chem. 1964, 1, 76).

A still another class of compounds of formula (I) of the present invention wherein R₆ represents NRaRb in which Ra and Rb represent both hydrogen, R₁-R₅ and R₇-R₁₀ are as defined above, i.e. compounds of formula (IX)

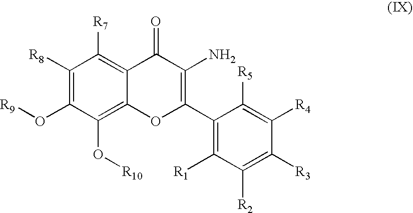

can be prepared by a process which comprises:

(A) reduction of the corresponding nitro derivative of formula (XII)

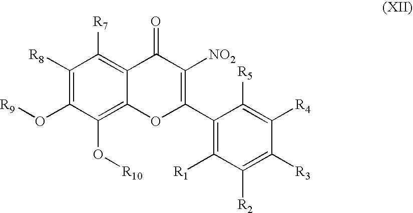

wherein R₁-R₅ and R₇-R₁₀ are as defined above under hydrogenolytic conditions. The reduction is preferably carried out under hydrogenolysis, by using hydrogen in the presence of a suitable catalyst such as, e.g. 10% Pd/C, in a suitable organic solvent such as, e.g., THF, dioxane or ethanol, preferably in dioxane.

A compound of formula (XII) can be obtained by a process which comprises: condensing an aldehyde of formula (XIII) wherein R₇-R₁₀ are as defined above with a compound of formula (XIV) wherein R₁-R₅ are as defined above to get a compound of formula (XV) wherein R₁-R₅ and R₇-R₁₀ are as defined above; oxidizing the compound of formula (XV) to the corresponding keto derivative of formula (XVI) wherein R₁-R₅ and R₇-R₁₀ are as defined above; eliminating Cl from the compound of formula (XVI) to obtain the compound of formula (XII), i.e. a compound of formula (I) wherein R₁-R₅ and R₇-R₁₀ are as defined above and R₆ is nitro following, for example, the method reported in the literature (see Eur. J. Med. Chem. 1997, 32, 71), according to Scheme 5 below.

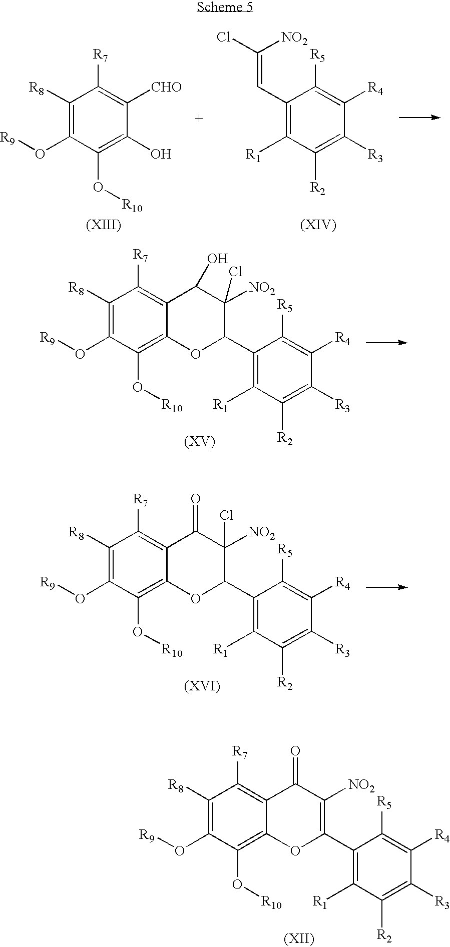

Typical reaction conditions for the condensation of a compound of formula (XIII) with a compound of formula (XIV) involve a suitable organic solvent, such as, e.g., DCM (methylene chloride), THF, dioxane, acetonitrile or 1,2-dimethoxyethane, preferably THF, in the presence of a base, such as, e.g., triethylamine, pyridine, or diisopropylethylamine, preferably triethylamine, at temperatures ranging from −20 to 50° C., most preferably room temperature, for a period of time ranging from 6 to 72 hrs. Oxidation of a compound of formula (XV) to obtain a compound of formula(XVI) can be performed with an oxidizing agent such as, e.g. PCC (pyridinium chlorochromate), in a suitable organic solvent such as, e.g. methylene chloride, at temperatures ranging from room temperatures to 100° C., typically 50° C. Reaction time might range from 8 to 48 hrs and preferred reaction conditions might involve the use of an ultrasound bath. Subsequent reaction of a compound of formula (XVI) to get a compound of formula (XII) is carried out in a proper organic solvent such as, e.g., THF, dioxane or pyridine, preferably THF, in the presence of a base such as, e.g., TEA, diisopropylethylamine or DBU (1,8-diazabicyclo[5.4.0]undec-7-ene), preferably DBU, at temperatures ranging from −20 to 50° C., most typically at room temperature. Reaction time might range from 30′ to 12 hrs.

Depending on the substitution pattern on the flavone skeleton, it may be important to add aqueous acids, such as for example hydrochloric acid, to the reaction mixture to preserve such substitution intact (like halogen substituents).

Intermediates of formula (XIV) necessary for the first step as depicted in Scheme 5 might be prepared through condensation of the corresponding aldehyde (XVII) wherein R₁-R₅ are as defined above with bromonitromethane, according to Scheme 6 below:

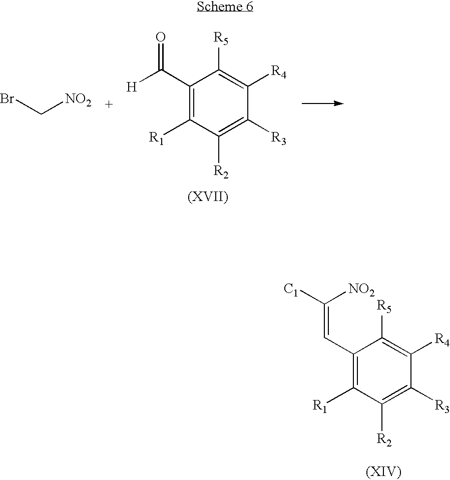

The condensation between a compound of formula (XVII) and bromonitromethane can be carried out, for example, in the presence of dimethylammonium chloride and potassium fluoride, in a suitable organic solvent such as, e.g., benzene, toluene or xylene, preferably xylene with the use of a Dean-Stark apparatus. Temperature ranges from 50 to 150° C., typically being the reflux temperature of the solvent for 1 to 24 hrs.

Alternatively, a compound of formula (IX) can be prepared by a process, which comprises:

(B) cyclization of an azido derivative of formula (XVIII)

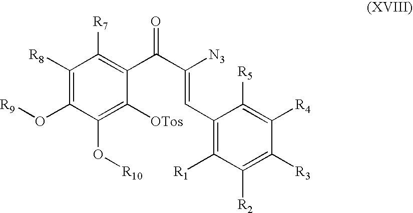

wherein R₁-R₅ and R₇-R₁₀ are as defined above, according to the method reported in the literature (see J. Med. Chem. 1991, 34, 736). Said method comprises: condensing an aldehyde of formula (XVII) wherein R₁-R₅ are as defined above with a derivative of formula (XXII) wherein R₇-R₁₀ are as defined above to yield the azido compound (XVIII); and rearranging the compound of formula (XVIII) to the expected amino derivative of formula (IX), as reported in the Scheme 7 below, part b). In scheme 7 part a), the preparation of the necessary intermediates for the steps in part b) is reported.

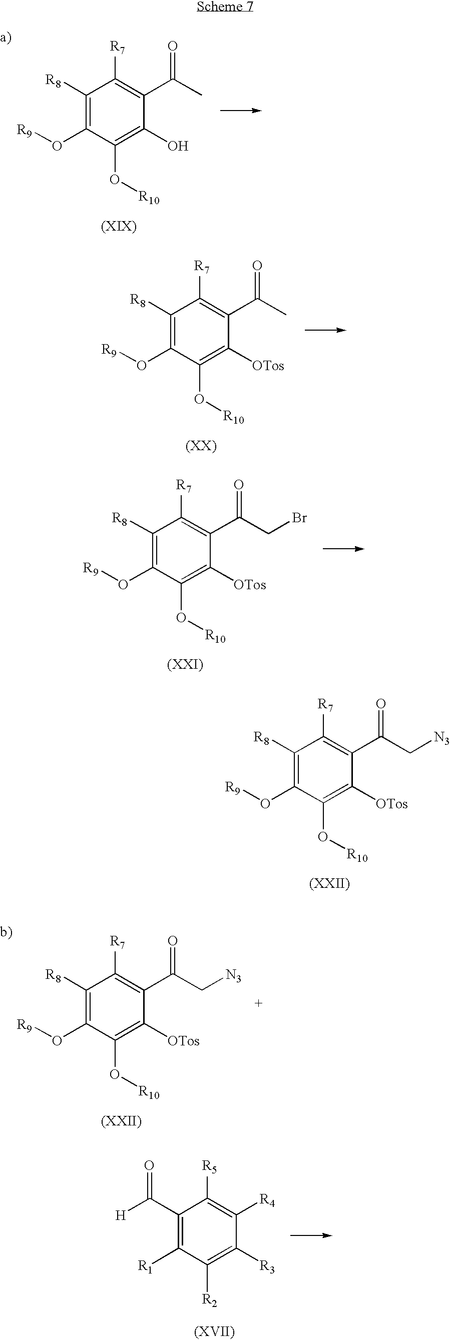

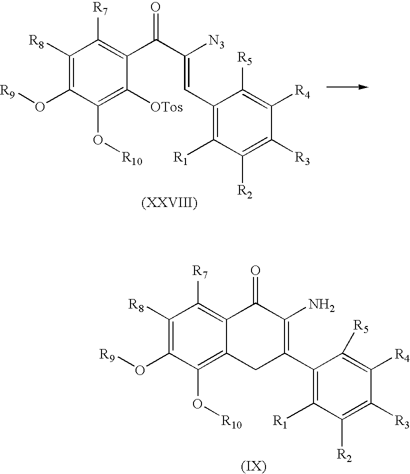

In particular, a compound of formula (XIX) wherein R₇-R₁₀ are as defined above is transformed to the corresponding tosylate of formula (XX) wherein R₇-R₁₀ are as defined above by using standard reaction conditions. In particular, it can be treated with p-Tolylsulfonyl chloride, in the presence of a base such as, e.g., potassium carbonate, sodium carbonate, pyridine or triethylamine, in a suitable organic solvent, such as, e.g., acetone, THF, acetonitrile, dioxane or methylene chloride, at temperatures ranging from −20° C. to 150° C., for 1 to 48 hrs. The compound of formula (XX) is then treated with a brominating agent to yield compound (XXI) wherein R₇-R₁₀ are as defined above. In particular, the compound of formula (XX) can be treated with dioxane bromide, in a proper organic solvent like ether, dioxane and similar, at temperatures ranging from −10° C. to reflux temperature for a time ranging from 1 to 72 hrs. The Compound of formula (XXI) is then transformed to the azido derivative of formula (XXII) wherein R₇-R₁₀ are as defined above, by the use of NaN₃ in a suitable organic solvent such as, e.g., DMSO, DMF or acetonitrile, at temperatures ranging from −20° C. to 20° C.

In scheme 7 part b), condensation of the azido compound of formula (XXII) and the aldehyde of formula (XVII) is depicted to yield the necessary intermediate of formula (XVIII) wherein R₁-R₅ and R₇-R₁₀ are as defined above, which is finally rearranged to the compound of formula (IX). In particular, the compound of formula (XXII) and the compound of formula (XVII) are treated with a proper reagent such as, e.g., piperidinium acetate, piperidinium tosylate or piperidinium hydrochloride, in a suitable organic solvent such as, e.g., ethanol, methanol, THF or methylene chloride, for 5 to 48 hrs, at temperatures ranging from −20° C. to 70° C., preferably at room temperature. Finally the compound of formula (XVIII) is treated with a base such as, e.g., sodium hydroxide or potassium hydroxide, in a proper organic solvent such as, e.g., ethanol, methanol or buthanol, for 30′ to 24, hrs, at temperatures ranging from 0° C. to 50° C., preferably at room temperatures.

A still further class of compounds of formula (I) wherein R₆ represents NRₐR_{b}, in which each of Ra and Rb represents, independently, C₁-C₆ alkyl, C₁-C₄-acyl, aroyl, C₁-C₆ alkylsulphonyl or arylsulphonyl, i.e. compounds of formula (X) and (XI)

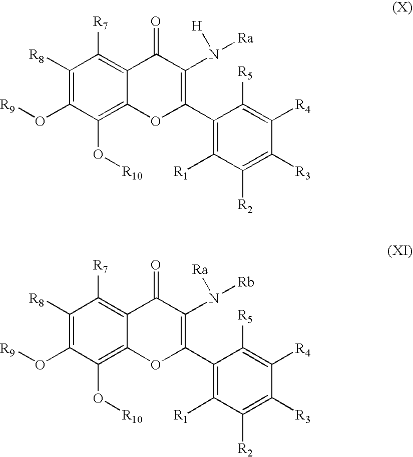

can be obtained by a process which comprises: treating a compound of formula (IX) as defined above with a suitable C₁-C₆ alkyl, C₁-C₄ acyl, C₁-C₆ alkylsulphonyl or arylsulphonyl halide, as known in the literature (see, for instance, March J., Advanced Organic Chemistry, Wiley Interscience), provided that other potentially interfering groups (like amino or hydroxy groups) are properly protected.

A compound of formula (II) can be obtained by a process which comprises:

A′) treatment of a compound of formula (XXIII)

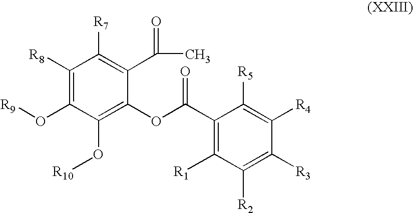

wherein R₁-R₅, and R₇-R₁₀ are as defined above, under basic conditions, as described in the literature (see, for example, Baker-Venkataraman rearrangement, J. Chem. Soc. 1933, 1381; J. Chem, Soc. 1934, 1767), according to the Scheme 8 below.

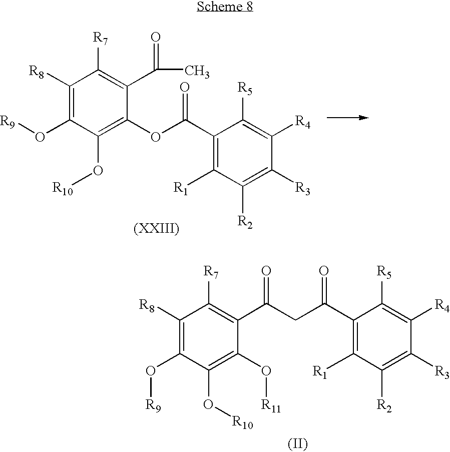

Typically in a compound of formula (II) formed through the route depicted above, R₁₁ is hydrogen. These reaction conditions preferentially involve treatment of a compound of formula (XXIII) either with an inorganic or with an organic base in a proper solvent, for example with potassium hydroxide or sodium hydroxide in pyridine, with potassium carbonate in isopropanol, or with sodium hydride (NaH) in a suitable solvent, such as, e.g., THF, DMF, DMSO or dioxane.

A compound of formula (XXIII) can be prepared by reacting a compound of formula (XXVI) wherein R₇-R₁₀ are as defined above, with a compound of formula (XXIX) wherein R₁-R₅ are as defined above and X represents a suitable leaving group, such as, e.g. halogen, preferably chlorine, as reported in the Scheme 8a below.

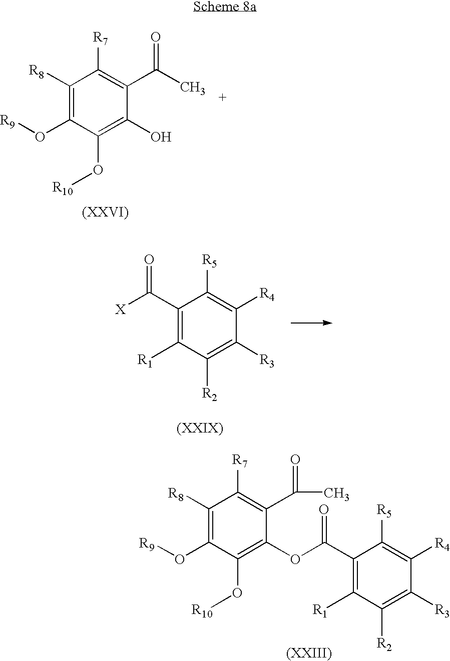

The reaction of a compound of formula (XXVI) and a compound of formula (XXIX) involves either an inorganic or an organic base, for example potassium carbonate, sodium carbonate, cesium carbonate, triethylamine or pyridine, in a suitable solvent, such as, e.g., acetone, THF, methylene chloride, dioxane or pyridine.

Compound of formula (II) can be alternatively obtained by a process, which comprises:

B′) condensing a compound of formula (XXIV) wherein R₇-R₁₀, and R₁₁ are as defined in formula (I) and R″ represents C₁-C₆ alkyl, preferably methyl or ethyl, with a compound of formula (XXV) wherein R₁-R₅ are as defined above, according to the Scheme 9 below.

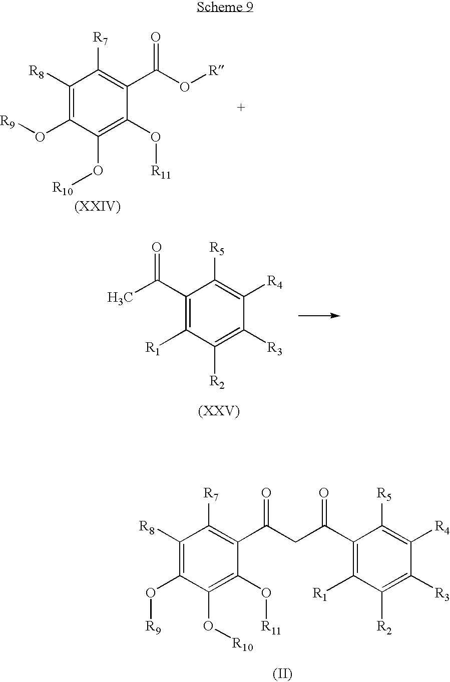

Typically a compound of formula (II) is obtained treating a compound of formula (XXIV) with a compound of formula (XXV) in the presence of a base (Claisen condensation). In particular, suitable reaction conditions may include the use of an inorganic base, like sodium hydride, in a proper organic solvent such as, e.g., THF, dioxane, DMF, DMSO or N,N-dimethylacetamide, under temperature ranging from −20° to reflux temperature.

A compound of formula (II) wherein R₁₁ represents hydrogen, i.e. a compound of formula (XXVIII)

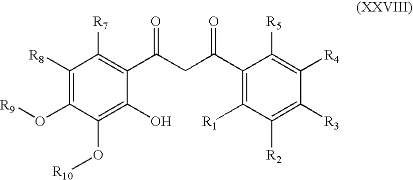

can be prepared by a process which comprises: reacting a compound of formula (XXVI) wherein R₇-R₁₀ are as defined above, with a compound of formula (XXVII) wherein R₁-R₅ are as defined above in a suitable solvent such as, e.g. THF, using a base such as, e.g., LiHMDS, NaHMDS or KHMDS, at temperature ranging from −78° C. to reflux temperature, according to the Scheme 10 below, where in case R₁-R₅ are hydroxyls, they are protected, for example as trialkylsilylethers, as described in the literature (see, for example, J. Org. Chem. 1991, 56, 4884).

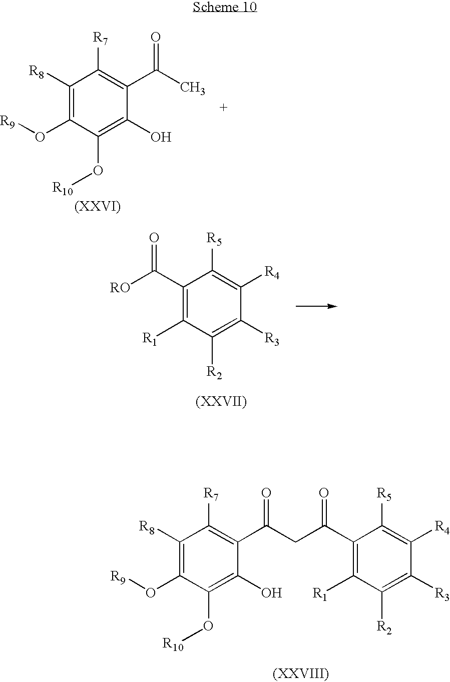

When in a compound of formula (I) R₃ and R₄, taken together, represent a 5 or 6 membered fused ring system having the formula —Y—(y)—X—(x)—Z—, these compounds of formula (I) can be obtained according to the previous Scheme 1 as already described, starting from suitably substituted precursors.

Particular cases of compounds of formula (I) can be also obtained according to other methods. For example particular cases of compounds of formula (I) wherein R₁, R₂ and R₁-R₈ are hydrogens, R₉ and R₁₀ are as defined above and R₃ and R₄, represent optionally substituted amino groups or R₃ and R₄, taken together, represent a 5 or 6 membered fused ring system having the formula

—Y—(y)—X—(x)—Z—

wherein

(ii′) X represents CR, —(y)— represents a double bond, —(x)— represents a single bond, Y represents N, Z represents NR′, R represents hydrogen or C₁-C₆ alkyl optionally substituted with C₁-C₆ dialkylamino and R′ represents hydrogen, C₁-C₆ alkyl, or C₁-C₆ acyl;

(iii′) X represents C—OR, —(y)— represents a double bond, —(x)— represents a single bond, Y represents N, Z represents NR′, and both R and R′ represent hydrogen;

(iv′) X represents C═O, —(y)— represents a single bond, —(x)— represents a single bond, Y and Z represents NR′ wherein R′ represents hydrogen;

(v′) X represents C—SR, —(y)— represents a double bond, —(x)— represents a single bond, Y represents N, Z represents NR′ and both R and R′ represent hydrogen;

(vi′) X represents C═S, —(y)— represents a single bond, —(x)— represents a single bond, Y and Z represent NR′ wherein R′ represents hydrogen;

(vii′) X represents N, —(y)— represents a double bond, —(x)— represents a single bond, Y represents N, Z represents NR′ wherein R′ represents hydrogen;

(viii′) X represents O═C—C═O, —(y)— represents a single bond, —(x)— represents a single bond, Y and Z represent NR′ wherein R′ represents hydrogen;

(ix′) when X represents RO—C—C═O, —(y)— represents a double bond, —(x)— represents a single bond, Y represents N, Z represents NR′ and and both R and R′ represent hydrogen;

(x′) X represents RO—C—C—OR, —(y)— represents a double bond, —(x)— represents a double bond, Y and Z are N, where R represents hydrogen,;

(xi′) X represents CH₂—CO, —(y)— represents a single bond, —(x)— represents a single bond, Y represents NR, Z represents NR′ and each of R and R′ represents, independently, hydrogen, C₁-C₆ alkyl or C₁-C₆ acyl;

(xii′) X represents CH₂—C—OR′, —(y)— represents a single bond, —(x)— represents a double bond, Y represents NR, Z represents N and each of R and R′ represents, independently, hydrogen, C₁-C₆ alkyl or C₁-C₆ acyl;

(xiii′) X represents CO—CH₂, —(y)— represents a single bond, —(x)— represents a single bond, Y represents NR′, Z represents NR and each of R and R′ represents, independently, hydrogen, C₁-C₆ alkyl or C₁-C₆ acyl; or

(xiv′) X represents R′O—C—CH₂, —(y)— represents a double bond, —(x)— represents a single bond, Y represents N, Z represents NR and each of R and R′ represents, independently, hydrogen, C₁-C₆ alkyl or C₁-C₆ acyl;

can be obtained according to the methods reported in schemes 11, 12 and 13, starting from an intermediate of formula (XXIII) wherein R₁, R₂, R₅, R₇ and R₈ are hydrogens, R₉ and R₁₀ are as defined above, R₃ represents acetylamino and R₄ is nitro, i.e. an intermediate of formula (XXXI).

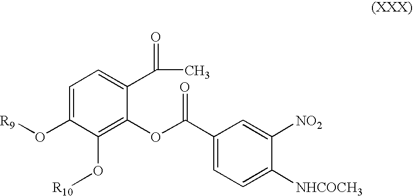

In particular, an intermediate of formula (XXX) can be transformed as represented in Scheme 11 below into a compound of formula (XXXI) wherein R₉ and R₁₀ are as defined above, that in turn can be cyclized to a compound of formula (I) wherein R₁, R₂, R₅, R₆, R₇ and R₈ are hydrogen, R₉ and R₁₀ are as defined above, R₃ is acetylamino and R₄ is nitro, i.e. a compound of formula (XXXII). The acetyl group from a compound of formula (XXXII) can be removed to yield the corresponding amino derivative of formula (I), i.e. a compound of formula (XXXIII) wherein R₉ and R₁₀ are as defined above, and the nitro group still present on the compound of formula (XXXIII) can be reduced under standard conditions to afford a compound of formula (I) wherein R₁, R₂, R₅, R₆, R₇ and R₈ are hydrogen, R₉ and R₁₀ are as defined above, R₃ and R₄ are amino, i.e. a compound of formula (XXXIV), according to methods known in the literature (see, for example, Eur. J. Med. Chem. 1995, 30, 561).

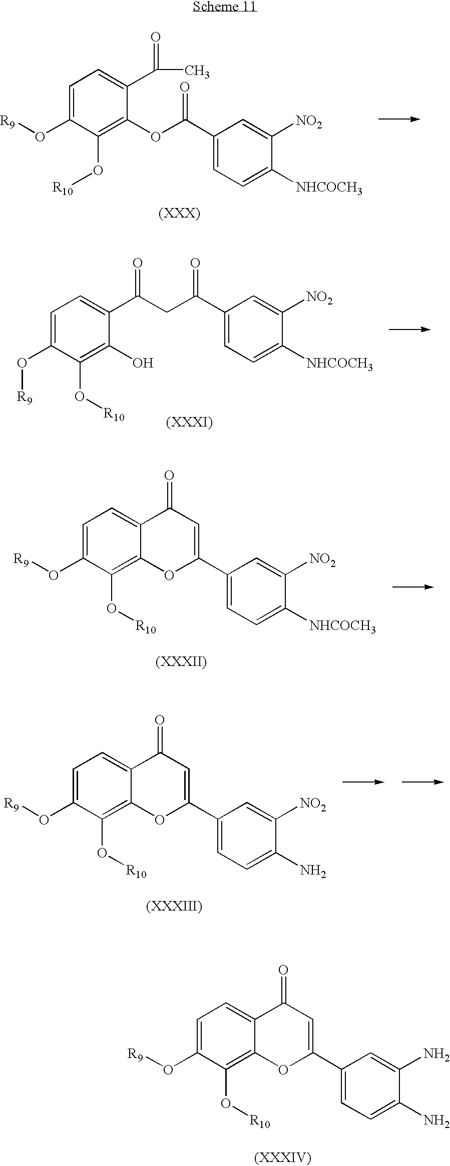

Compounds of formula (XXX) can be prepared according to the methods described in Scheme 8a, while a compound of formula (XXXI) can be obtained via Baker-Venkataraman rearrangement according to the procedures reported in Scheme 8. A compound of formula (XXXII) can be prepared as described in Scheme 1, preferably by the use of anhydrous sodium acetate in glacial acetic acid under refluxing conditions. A compound of formula (XXXIII) can be prepared by hydrolysis under acidic conditions, like aqueous hydrochloric acid, neat sulphuric acid or in the presence of a suitable organic solvent, such as ethanol and the like, at temperatures ranging from room to refluxing temperatures for 1 to 24 hrs, preferably under reflux for 5 hrs. A compound of formula (XXXIV) can be obtained under reduction conditions suitable for the nitro group, such as catalytic hydrogenation in the presence of palladium on charcoal, metals in acidic conditions, like Fe, Sn, and Zn, formic acid with palladium catalyst, Zn in the presence of hydrated NiCl₂ in methanol with or without a proper cosolvent, such as DMF and the like. Preferred conditions are Zn with hydrated NiCl₂ in methanol/DMF at 70° C. for 3 hrs.

Compounds of formula (XXXIV) can be used to obtain several other compounds of formula (I) as depicted in Scheme 12 below. As reported in Scheme 12 in the presence of several condensing agents or acylating agents under different reaction conditions, the compounds of the scheme, wherein A in formulas (XXXIX) and (XL) represents oxygen and sulphur respectively, can be obtained as depicted and in all other conceivable tautomeric forms as well.

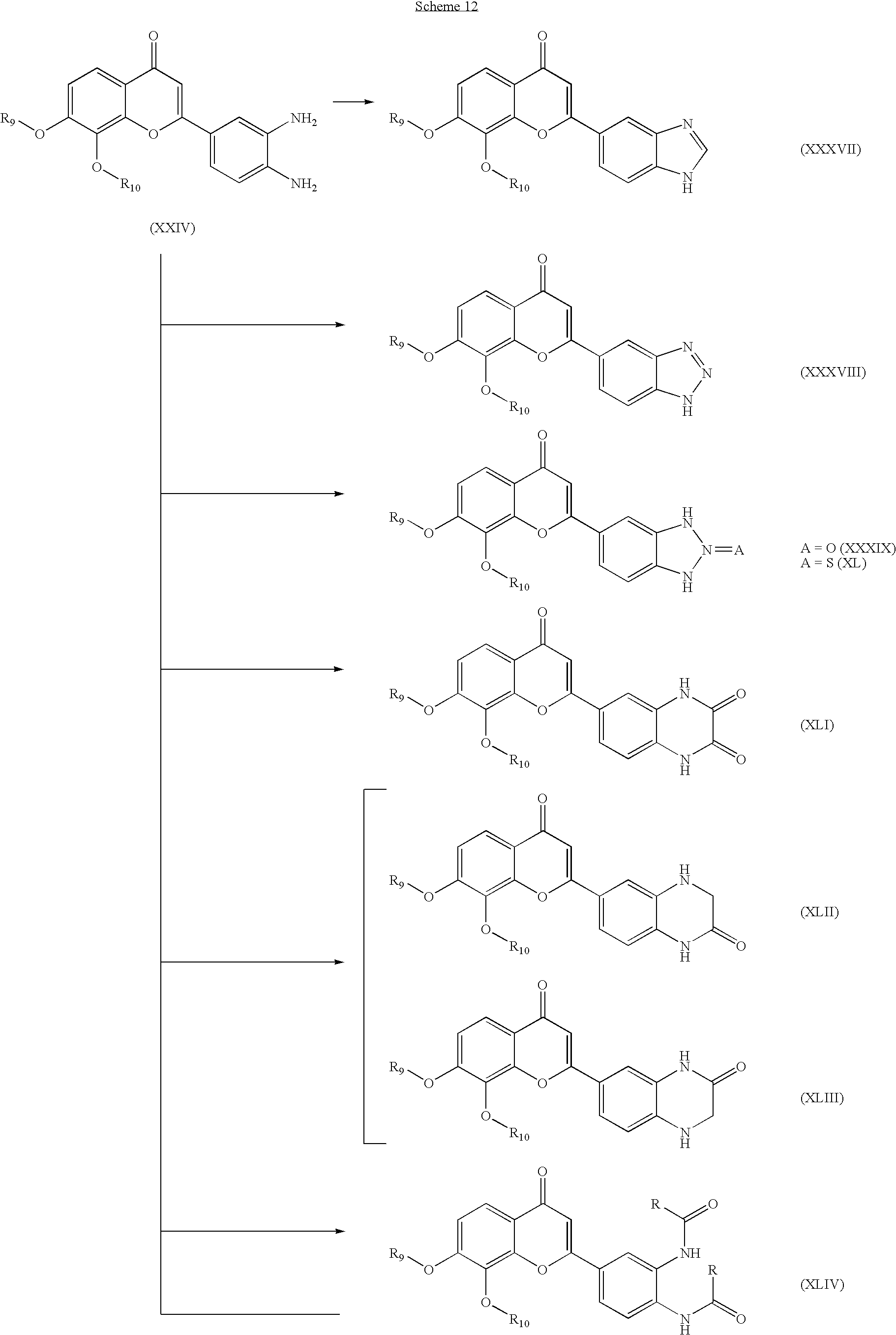

In particular a compound of formula (I) wherein R₁, R₂, R₅-R₈ are hydrogen, R₉ and R₁₀ are as defined above and R₃ and R₄, taken together, represents a group —Y—(y)—X—(x)—Z— wherein X represents CR, —(y)— represents a double bond, —(x)— represents a single bond, Y represents N, Z represents NR′, R and R′ represent hydrogen, i.e. a compound of formula (XXXVII), can be obtained by condensing a compound of formula (XXXIV) with several agents such as, e.g., formic acid in aqueous hydrochloric acid, or trimethyl orthoformate, triethyl orthoformate, dichloromethyl methyl ether, in a suitable organic solvent such as, e.g., THF or dioxane, at temperatures ranging from room refluxing temperatures for 1 to 24 hrs.

A compound of formula (I) wherein R₁, R₂, R₅-R₈ are hydrogen, R₉ and R₁₀ are as defined above and R₃ and R₄, taken together, represent a group —Y—(y)—X—(x)—Z— wherein X represents N, —(y)— represents a double bond, —(x)— represents a single bond, Y represents N, Z represents NR′ and R′ represents hydrogen, i.e. a compounds of formula (XXXVIII), can be prepared by treating a compound of formula (XXXIV) with sodium nitrite in a proper solvent system like water and glacial acetic acid, at temperatures ranging from −10° C. to 20° C., for 30′ to 10 hrs, according to the methods of the literature (see U.S. Pat. No. 4,299,965).

A compound of formula (I) wherein R₁, R₂, R₅-R₈ are hydrogen, R₉ and R₁₀ are as defined above and R₃ and R₄, taken together, represent a group —Y—(y)—X—(x)—Z— wherein X represents C═O, —(y)— represents a single bond, —(x)— represents a single bond, Y represents NR′, Z represents NR′ and R′ represents hydrogen, i.e. a compound of formula (XXXIX), can be prepared condensing a compound of formula (XXXIV) with carbonyl diimidazole in THF, triphosgene, phosgene, and ethyl chloroformate in a suitable organic solvent like THF, methylene chloride or dioxane in presence of inorganic or organic base, at temperature ranging from −10° C. to room temperature, for 30′ to 24 hrs.

A compound of formula (I) wherein R₁, R₂, R₅-R₈ are hydrogen, R₉ and R₁₀ are as defined above and R₃ and R₄, taken together, represent a group —Y—(y)—X—(x)—Z— wherein X represents C═S, —(y)— represents a single bond, —(x)— represents a single bond, Y represents NR′, Z represents NR′ and R′ represents hydrogen, i.e. a compound of formula (XL), can be obtained condensing a compound of formula (XXXIV) with 1,1′-thiocarbonyldiimidazole, thiophosgene, potassium xanthate, thiourea in a suitable organic solvent like pyridine, DMF, ethanol, methylene chloride, in presence of inorganic or organic base like sodium carbonate, potassium carbonate, triethylamine, pyridine, at temperature ranging from −10° C. to room temperature, for 1 to 48 hrs.

A compound of formula (I) wherein R₁, R₂, R₅-R₈ are hydrogen, R₉ and R₁₀ are as defined above and R₃ and R₄, taken together, represent a group —Y—(y)—X—(x)—Z— wherein X represents O═C—C═O, —(y)— represents a single bond, —(x)— represents a single bond, Y and Z represent NR′ wherein R′ represents hydrogen, i.e. a compound of formula (XLI), can be prepared by treating a compound of formula (XXXIV) with oxalyl chloride, diethyl oxalate, oxalic acid in a suitable solvent like pyridine, THF, dioxane, aqueous hydrochloric acid, at temperature ranging to room temperature to reflux, for 1 to 24 hrs.

A compound of formula (I) wherein R₁, R₂, R₅-R₈ are hydrogen, R₉ and R₁₀ are as defined above and R₃ and R₄, taken together, represent a group —Y—(y)—X—(x)—Z— wherein X represents —CH₂—CO, —(y)— represents a single bond, —(x)— represents a single bond, Y represents NR, Z represents NR′ and R and R′ represent hydrogen, i.e. a compounds of formula (XLII), and a compound of formula (I) wherein R₁, R₂, R₅-R₈ are hydrogen, R₉ and R₁₀ are as defined above and R₃ and R₄, taken together, represents a group —Y—(y)—X—(x)—Z— wherein X represents CO—CH₂, —(y)— represents a single bond, —(x)— represents a single bond, Y represents NR′, Z represents NR and R and R′ represent hydrogen, i.e. a compound of formula (XLIII), can be obtained condensing a compound of formula (XXXIV) with chloroacetic acid in water in presence of inorganic base like potassium hydroxide or sodium hydroxide, chloroacetyl chloride in a suitable organic solvent like pyridine, toluene, THF, dioxane, at temperature ranging from room temperature to reflux, for 1 to 24 hrs. The mixture of regioisomeric forms can be separated by usual methods.

A compound of formula (I) wherein R₁, R₂, R₅-R₈ are hydrogen, R₉ and R₁₀ are as defined above and R₃ and R₄, represent C₁-C₄ acylamino, i.e. a compound of formula (XLIV) wherein R is C₁-C₄ alkyl, can be prepared by treating the diamino derivative (XXXIV) with acetic anhydride, acetyl chloride and the like in a suitable organic solvent like THF, dioxane, methylene chloride, in presence of inorganic or organic base like potassium carbonate, sodium carbonate, triethylamine, pyridine, at temperature ranging from room temperature to reflux, for 1 to 10 hrs. Preferred conditions are acetyl chloride in THF with the presence of triethylamine at room temperature for 5 hrs.

A compound of formula (I) wherein R₁, R₂, R₅-R₈ are hydrogen, R₉ and R₁₀ are as defined above, and R₃ and R₄, taken together, represent a group —Y—(y)—X—(x)—Z— wherein X represents CR, —(y)— represents a double bond, —(x)— represents a single bond, Y represents N, Z represents NR′ wherein R′ is hydrogen and R represents methyl, i.e. a compound of (XXXVI), can be obtained as reported in scheme 13 from a compound of formula (XXXII). A compound of formula (XXXII) in fact can be reduced with similar methods described for a compound of formula (XXXIII) in Scheme 11 above, and thus obtaining a compound of formula (I) wherein R₁, R₂, R₅-R₈ are hydrogen, R₉ and R₁₀ are as defined above, R₃ is acetylamino and R₄ is amino, i.e. a compound of formula (XXXV), that can be cyclized to a compound of formula (XXXVI) under acidic conditions, as depicted in Scheme 13 below.

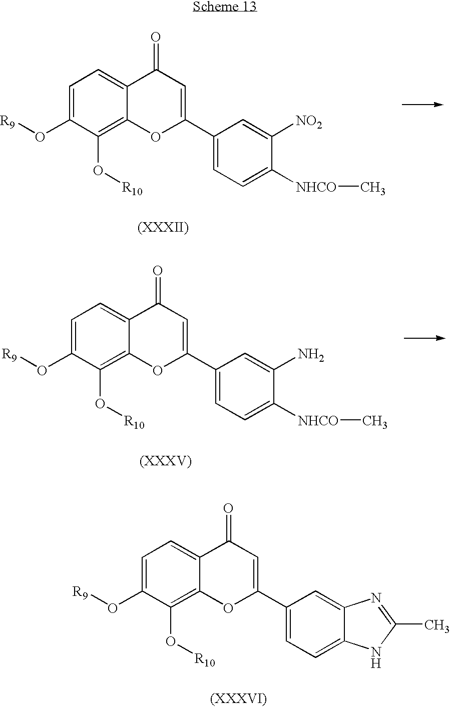

In particular a compound of formula (XXXVI) can be obtained by treating a compound of formula (XXXV) with acids, like diluted aqueous hydrochloric acid at temperature ranging from 40° C. to reflux preferably under refluxing conditions.

The compounds of formula (I), (Ia), (Ib) and (Ic) are herein defined as the “compounds of the present invention”, the “compounds of the invention” and/or the “active principles of the pharmaceutical compositions of the invention”.

The compounds of the invention can be administered in a variety of dosage forms, e.g. orally, in the form of tablets, capsules, lozengers, liquid solutions or suspensions; rectally, in the form of suppositories; parenterally, e.g. intramuscularly, intravenously, intradermally or subcuteneously; or topically.

The dosage depends upon, for example, the compound of the invention employed, the age, weight, condition of the patient and administration route; specific dosage regimens may be fit to any particular subject on the basis of the individual need and the professional judgement of the person administering or supervising the administration of the aforesaid compounds. For example, the dosage adopted for the administration to adult humans may range from 0.001 to 100 mg of compound of the invention per kg of body weight; a particularly preferred range may be from 0.1 to 10 mg of compound of the invention per kg of body weight.

The dosages may be administered at once or may be divided into a number of smaller doses to be administered at varying intervals of time.

As already mentioned above, pharmaceutical compositions containing, as an active ingredient, a compound of the present invention or a pharmaceutically acceptable salt thereof in association with a pharmaceutically acceptable excipient, are also within the scope of the present invention.

These pharmaceutical compositions contain an amount of active ingredient, which is therapeutically effective to display, for example, antileukemic and/or antitumor activity.

There may also be included as a part of the pharmaceutical compositions according to the invention, pharmaceutically acceptable binding agents and/or adjuvant materials. The active ingredients may also be mixed with other active principles, which do not impair the desired action and/or supplement the desired action.

The pharmaceutical compositions containing the compounds of the invention are usually prepared following conventional methods and may be administered in a pharmaceutically suitable form.

For example, the solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents, e.g. starches, arabic gums, gelatin, methylcellulose, microcrystalline cellulose, carboxymethylcellulose or polyvinyl pyrrolidone; diaggregating agents, e.g. a starch, alginic acid, alginates or sodium starch glycolate; effervescing mixtures; dyestuffs; sweetening agents, e.g. sucrose or saccharin; flavouring agents, e.g. peppermint, methylsalicylate or orange flavouring; wetting agents, such as lecithin, polysorbates, laurylsulphates; and, in general, non-toxic and pharmacologically inactive substances used in pharmaceutical formulations.

When the dosage unit form is a capsule, it may contain, in addition to material of the above type, a liquid carrier such as, e.g., a fatty oil.

Said pharmaceutical preparations may be manufactured in known manner, for example, by means of mixing, granulating, tabletting, sugar-coating or film-coating processes. The liquid dispersions for oral administration may be, e.g. syrups, emulsions and suspensions.

The syrups may contain as carrier, for example, saccharose or saccharose with glycerine and/or mannitol and/or sorbitol; in particular, a syrup to be administered to diabetic patients can contain as carriers only products not metabolizable to glucose, or metabolizable in very small amount to glucose, for example sorbitol.

The suspensions and the emulsions may contain as carrier, for example, a natural gum, agar, sodium alginate, pectin, methylcellulose, carboxymethylcellulose, or polyvinyl alcohol.

The suspensions or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride.

The solutions for intravenous injections or infusions may contain as carrier, for example, sterile water, or preferably they may be in the form of sterile, aqueous, isotonic saline solution.

The solutions or suspensions for parenteral therapeutic administration may also contain antibacterial agents, such as benzyl alcohol or methyl parabens; antioxidants, such as ascorbic acid or sodium bisulphite; chelating agents, such as ethylenediamine tetraacetic acid; buffers, such as acetates, citrates or phosphates and agents for the adjustment of tonicity, such as sodium chloride or dextrose. The parenteral preparation can be enclosed in ampoules, disposable syringes or multiple dose vials made of glass or plastic.

The suppositories may contain together with the active compound a pharmaceutically acceptable carrier, e.g., coca-butter, polyethylene glycol, a polyoxyethylene sorbitan fatty acid ester surfactant or lecithin.

Compositions for topical application, such as, e.g., creams, lotions or pastes, may be, e.g., prepared by admixing the active ingredient with a conventional oleaginous or emulsifying excipient.

Biological Activity

Flash Plate-Based Assay

The telomerase activity of the compounds has been evaluated using a Flash Plate-based assay. The method proved to be sensitive, accurate and able to reproducibly identify compounds that inhibit telomerase activity in a dose-dependent manner.

Briefly the assay mixture is constituted of:

telomerase enzyme diluted in a buffer, the composition of which has been selected to maintain the enzyme activity stable along the duration of the assay.

dNTPs, deoxynucleotides 5′-triphosphate.

biotinylated oligo as primer.

increasing concentrations of test compounds/positive control.

After two hours of incubation at 37° degrees the telomeric repeats added by telomerase are evaluated by hybridization in solution with a 3′-radioactive labeled short oligonucleotide probe.

The extent of hybridization is then quantitated by transferring the reaction mixture in a streptavidin-coated flash plate, where the binding between biotin and streptavidin occurs.

The telomerase activity is proportional to the radioactivity measured and the inhibitory activity of the compounds is evaluated as IC₅₀ using the Sigma Plot fit program.

With the above-described method IC₅₀ values of the compounds of the present invention were determined.

The results relative to a representative selection of compounds of the invention are shown in Table 1.

TABLE 1

| Compound | IC₅₀ (μM) |
|---|---|
| 1 | 33 |
| 2 | 3 |
| 3 | 0.3 |
| 4 | 25.3 |
| 5 | 34 |
| 7 | 12 |
| 14 | 15 |
| 24 | >>40 |
| 37 | 0.8 |
| 38 | >>40 |
| 39 | 0.13 |
| 41 | 0.6 |
| 45 | 30 |
| 46 | 14.3 |
| 49 | 0.34 |
| 50 | 5.4 |
| 51 | 7.4 |
| 52 | 3.6 |
| 58 | >>40 |
| 61 | 2.2 |
| 65 | >>40 |
| 67 | 19 |
| 77 | 2.6 |
| 78 | 7.8 |
| 79 | 11.7 |
| 87 | 8.7 |
| 90 | 0.64 |

The data reported in Table 1 clearly show the activity of the compounds according to the invention as telomerase inhibitors.

A human or animal body may thus be treated by a method, which comprises the administration thereto of a pharmaceutically effective amount of a compound of formula (I) or a salt thereof. The condition of the human or animal can thereby be improved.

Combination chemotherapy using two or more anti-cancer drugs to treat malignant tumors in humans is currently in use in research and in the clinic.

The anti-cancer drugs may be, for example, topoisomerase inhibitors, antimetabolites, alkylating agents, antibiotics, antimicrotubule agents or anti-angiogenesis agents.

Combinations of drugs are administered in an attempt to obtain a synergistic effect on most cancers, e.g., carcinomas, melanomas, sarcomas, lymphomas and leukemias and to reduce or eliminate emergence of drug-resistant cells and to reduce side effects to each drug.

It is therefore a still further aspect of the present invention a combination therapy of a compound according to the invention with at least one other anti-cancer agent. The use of active substances together provides improved therapeutic effect than employing the single agents alone. Antineoplastic agents suitable for combination with the compounds of the present invention include, but are not limited to:

topoisomerase I inhibitors comprising,for example, epipodophyllotoxins such as, e.g. etoposide and teniposide; camptothecin and camptothecin derivatives including, e.g., irinotecan, SN-38, topotecan, 9-amino-camptothecin, 10,11-Methylenedioxy camptothecin and 9-nitro-camptothecin (rubitecan);

alkylating agents including nitrogen mustards such as, e.g., mechlorethamine, chlorambucil, melphalan, uracil mustard and estramustine; alkylsulfonates such as, e.g., busulfan improsulfan and piposulfan; oxazaphosphorines such as e.g., ifosfamide, cyclophosphamide, perfosfamide, and trophosphamide; platinum derivatives such as, e.g., oxaliplatin, carboplatin and cisplatin; nitrosoureas such as, e.g., carmustine, lomustine and streptozocin;

antimitotic agents including taxanes such as , e.g., paclitaxel and docetaxel; vinca alkaloids such as, e.g., vincristine, vinblastine, vinorelbine and vindesine; and novel microtubule agents such as, e.g., epothilone analogs, discodermolide analogs and eleutherobin analogs;

antimetabolites including purines such as , e.g., 6-mercaptopurine, thioguanine, azathioprine, allopurinol, cladribine, fludarabine, pentostatin, and 2-chloro adenosine; fluoropyrimidines such as, e.g., 5-FU, fluorodeoxyuridine, ftorafur, 5′-deoxyfluorouridine, UFT, S-1 and capecitabine; and pyrimidine nucleosides such as, e.g., deoxycytidine, cytosine arabinoside, 5-azacytosine, gemcitabine, and 5-azacytosine-arabinoside; antimetabolites (for example antifolates like methotrexate, fluoropyrimidines like 5-fluorouracil, purine and adenosine analogues, cytosine arabinoside;

hormones, hormonal analogues and hormonal antagonists including antiestrogens (for example tamoxifen, toremifen, raloxifene, droloxifene and iodoxyfene), progestogens (for example megestrol and acetate), aromatase inhibitors (for example anastrozole, letrazole, borazole and exemestane), antiprogestogens, antiandrogens (for example flutamide, nilutamide, bicalutamide and cyproterone acetate), LHRH agonists and antagonists (for example gosereline acetate and luprolide) and inhibitors of testosterone 5a-dihydroreductase (for example finasteride;

antitumor antibiotics including anthracyclines and anthracenediones such as, e.g., doxorubicin, daunorubicin, epirubicin, idarubicin and mitoxantrone;

farnesyltransferase inhibitors including, for example, SCH 44342, RPR 113228, BZA 5B and PD 161956;

anti-invasion agents (for example metalloproteinase inhibitors such as, e.g., marimastat and inhibitors of urokinase plasminogen activator receptor functions);

inhibitors of growth factor (for example, EGF, FGF, platelet derived growth factor and hepatocyte growth factor) functions including growth factor antibodies, growth factor receptor antibodies, tyrosine kinase inhibitors and serine/threonine kinase inhibitors;

antiangiogenic agents such as, for example, linomide, inhibitors of integrin avβ3 function, angiostatin, razoxin, SU 5416, SU 6668, AGM 1470 (TNP-470), a synthetic analogue of fumagillin a naturally secreted product of the fungus Aspergillus fumigates fresenius, platelet factor 4 (endostatin), thalidomide, marimastat (BB-2516) and batimastat (BB-94); and

cell cycle inhibitors such as, e.g., flavopyridols.

In a further aspect of this invention, a method is provided for treating a cancer comprising administering to a patient in need of such treatment a therapeutically effective amount of a substituted benzopyranone as defined in formula (I) above or a pharmaceutically acceptable salt thereof and a therapeutically effective amount of at least another chemotherapeutic agent.

The following examples illustrate but do not limit the invention:

General

Compounds XIII, XVII, XIX, XXVI, XXIX, XXIV, XXV and XXVII can be prepared by a person skilled in the art starting from commercially available compounds and following known literature methods. Ethyl 2,3,4-trimethoxybenzoate can be prepared from the corresponding commercially available carboxylic acid by the method described in Org. Prep. Proceed. 1996, 28, 480-483, herein incorporated by reference. 3,4-Dimethoxy-2-hydroxyacetophenone, 2-(3,4-dihydroxyphenyl)-7,8-dihydroxy-4H-chromen-4-on, 2,3-dihydroxy-4-methoxy acetophenone and 2-hydroxy-3,4-dimethoxy acetophenone are commercially available compounds.

### EXAMPLE 1

1-(3-methoxyphenyl)-3-(2,3,4-trimethoxyphenyl)-1,3-propanedione

To a suspension of sodium hydride (60% in oil, washed with hexane, 320 mg, 8 mmol) in anhydrous dioxane (10 mL), under argon, a mixture of ethyl 2,3,4-trimethoxybenzoate (960 mg, 4 mmol) and 3-methoxyacetophenone (333 mg, 2.2 mmol) in anhydrous dioxane (10 mL) is added dropwise at room temperature. The reaction mixture is then stirred at reflux for 5 hours. After cooling, excess of sodium hydride is destroyed by addition of ethanol, the mixture acidified with 2N HCl, extracted with ethylacetate, washed with water, dried, and evaporated under reduced pressure. The crude reaction product is purified by flash chromatography (eluant: hexane/tetrahydrofuran 8:2) to yield 1-(3-methoxyphenyl)-3-(2,3,4-trimethoxyphenyl)-1,3-propanedione as a yellow solid. Yield: 33%.

¹H-NMR (400 Mhz, CDCl₃), ppm: 3.8-4.0 (12H, m), 7.0-7.2 (4H, m), 7.4-7.6 (4H, m).

By analogous procedure the following compounds were prepared:

1-(3-chlorophenyl)-3-(2,3,4-trimethoxyphenyl)-1,3-propanedione;

¹H-NMR (400 Mhz, CDCl₃), ppm: 3.8 (3H, s), 3.9 (3H, s), 7.0-7.2 (4H, m), 7.3 (1H, m), 7.6 (1H, d), 7.8 (1H, dd), 7.9 (1H, s); and

1-(3,4-dichlorophenyl)-3-(2,3,4-trimethoxyphenyl)-1,3-propanedione;

¹H-NMR (400 Mhz, CDCl₃), ppm: 3.8 (3H, s), 3.9 (3H, s), 7.0-7.2 (4H, m), 7.5 (1H, d), 7.8 (1H, dd), 7.9 (1H, s).

### EXAMPLE 2

2-(3-Chlorophenyl)-7,8-dihydroxy-4H-chromen-4-one (Compound 45)

A suspension of 1-(3-chlorophenyl)-3-(2,3,4-trimethoxyphenyl)-1,3-propanedione (110 mg, 0.29 mmol) in a mixture of hydriodic acid (57%, 4 mL) and glacial acetic acid (4 mL) is refluxed for 15 hours. After cooling the yellow precipitate is filtered and washed with acetic acid and water. The solid is suspended into an aqueous NaHSO₃ solution and stirred for 1 hour, filtered, washed with water and dried in vacuum to yield 2-(3-Chlorophenyl)-7,8-dihydroxy-4H-chromen-4-one as a white solid. Yield: 45%.

¹H-NMR (400 Mhz, DMSOd₆), ppm: 6.94 (1H, d, J=8.7 Hz), 6.95 (1H, s), 7.38 (1H, d, J=8.7 Hz), 7.58 (1H, dd, J=9.8, 7.8 Hz), 7.63 (1H, ddd, J=1.7, 1.7, 7.8 Hz), 8.08 (1H, ddd, J=1.7, 1.7, 7.8 Hz), 8.25 (1H, dd, J=1.7, 1.7 Hz).

By analogous procedure and starting from the appropriate 1,3-propanediones, the following compounds were prepared:

2-(3,4-Dichlorophenyl)-7,8-dihydroxy-4H-chromen-4-one (compound 46)

¹H-NMR (400 Mhz, DMSOd₆), ppm: 6.94 (1H, d, J=8.7 Hz), 7.00 (1H, s), 7.38 (1H, d, J=8.7 Hz), 7.82 (1H, d, J=8.5 Hz), 8.12 (1H, dd, J=2.1, 8.5 Hz), 8.46 (1H, d, J=2.1 Hz);

2-(3-fluoro-4-hydroxyphenyl)-7,8-dihydroxy-4H-chromen-4-one (compound 98);

2-(4-fluoro-3-hydroxyphenyl)-7,8-dihydroxy-4H-chromen-4-one (compound 99);

2-(4-fluorophenyl)-7,8-dihydroxy-4H-chromen-4-one (compound 43);

2-(3-fluorophenyl)-7,8-dihydroxy-4H-chromen-4-one (compound 44);

4-(7,8-dihydroxy-4-oxo-4H-chromen-2-yl)benzoic acid (compound 64); and

4-(7,8-dihydroxy-4-oxo-4H-chromen-2-yl)benzonitrile (compound 89).

### EXAMPLE 3

1-(5-Chloro-2-hydroxy-3,4-dimethoxyphenyl)ethanone

A solution of 3,4-dimethoxy-2-hydroxyacetophenone (200 mg, 1.02 mmol) and N-chlorosuccinimide (163 mg, 1.22 mg) in glacial acetic acid (5 mL) is stirred at 80° C. for 6 hours. After cooling the solution is diluted with water and extracted with ethylacetate. The organic phase is washed with water and brine, dried and evaporated under reduced pressure. The crude reaction product is purified by flash chromatography (eluant hexane/ethylacetate 9:1) to yield 1(5-chloro-2-hydroxy-3,4-dimethoxyphenyl)ethanone as yellowish solid. Yield: 61%.

¹H-NMR (400 Mhz, CDCl₃), ppm: 2.59 (3H, s), 3.91 (3H, s), 4.08 (3H, s), 7.52 (1H, s).

### EXAMPLE 4

6-Acetyl-4-chloro-2,3-dimethoxyphenyl 3,4-dimethoxybenzoate

To a solution of 1-(5-chloro-2-hydroxy-3,4-dimethoxyphenyl)ethanone (400 mg, 1.73 mmol) in anhydrous pyridine (5 mL), under argon, 3,4-dimethoxybenzoylchloride (520 mg, 2.59 mmol) is added in a period of 15 min. The mixture is stirred for 2 hours at room temperature, then acidified with 2N HCl, extracted with ethylacetate, washed with water, dried, and evaporated under reduced pressure. The crude reaction product is purified by flash chromatography (eluant hexane/ethylacetate 7:3) to yield 6-acetyl-4-chloro-2,3-dimethoxyphenyl 3,4-dimethoxybenzoate as white solid. Yield: 88%.

¹H-NMR (400 Mhz, DMSOd₆), ppm: 2.45 (3H, s), 3.78 (3H, s), 3.82 (3H, s), 3.88 (3H, s), 3.92 (3H, s), 7.18 (1H, d), 7.55 (1H, ds), 7.78-7.82 (2H, m).

By analogous procedure and starting from the properly substituted ethanones and aroyl chlorides, the following compounds were prepared:

6-Acetyl-2,3-dimethoxyphenyl 2,4-dimethoxybenzoate; purified by flash chromatography (eluant hexane/ethylacetate 1:1).

¹H-NMR (400 Mhz, CDCl₃), ppm: 2.40 (3H, s), 3.85-3.90 (12H, m), 6.60-6.80 (3H, m), 7.55 (1H, d), 8.10 (1H, d);

6-Acetyl-2,3-dimethoxyphenyl 4-(acetylamino)-3-nitrobenzoate; purified by flash chromatography (eluant hexane/ethylacetate 1:1).

¹H-NMR (400 Mhz, CDCl₃), ppm: 2.35 (3H, s), 2.45 (3H, s), 3.80 (3H, s), 3.95 (3H, s), 6.90 (1H, d), 7.65 (1H, d), 8.43 (1H, d), 9.00 (1H, d), 9.10 (1H, s), 10.60 (1H, brs);

6-acetyl-2,3-dimethoxyphenyl 3-fluoro-4-methoxybenzoate;

6-acetyl-2,3-dimethoxyphenyl 4-fluoro-3-methoxybenzoate;

6-acetyl-2,3-dimethoxyphenyl 3-fluorobenzoate;

6-acetyl-2,3-dimethoxyphenyl 4-fluorobenzoate;

6-acetyl-2,3-dimethoxyphenyl 4-cyanobenzoate

6-Acetyl-5-chloro-2,3-dimethoxyphenyl 3,4-dimethoxybenzoate;

6-Acetyl-2,3-dimethoxyphenyl 1,3-benzoxazole-5-carboxylate; and

6-Acetyl-2,3-dimethoxyphenyl 1,3-benzothiazole-5-carboxylate.

### EXAMPLE 5

1-(5-Chloro-2-hydroxy-3,4-dimethoxyphenyl)-3-(3,4-dimethoxyphenyl)-1,3-propanedione

To a solution of 6-acetyl-4-chloro-2,3-dimethoxyphenyl-3,4-dimethoxybenzoate (580 mg, 1.47 mmol) in anhydrous pyridine (5 mL), stirred at 50° C., powdered potassium hydroxide (125 mg, 2.25 mmol) is added. After 1 hour, the reaction mixture is cooled, acidified with 2N HCl, extracted with ethylacetate, washed with water, dried, and evaporated under reduced pressure. The crude reaction product is purified by flash chromatography (eluant hexane/ethylacetate 7:3) to yield 1-(5-chloro-2-hydroxy-3,4-dimethoxyphenyl)-3-(3,4-dimethoxyphenyl)-1,3-propanedione as yellow solid. Yield: 62%.

1H-NMR (400 Mhz, DMSOd₆), ppm: 3.80 (3H, s), 3.82-3.90 (6H, m), 3.88 (3H, s), 7.10 (1H, d), 7.25 (1H, s), 7.55 (1H, ds), 7.75 (1H, dd), 7.95 (1H, s), 11.40-11.60 (2H, m).

By analogous procedure and starting from the appropriate aryl benzoates, the following compounds were prepared:

1-(2,4-Dimethoxyphenyl)-3-(2-hydroxy-3,4-dimethoxyphenyl)-1,3-propanedione;

¹H-NMR (400 Mhz, DMSOd₆), ppm: 2.95 (2H, s), 3.80-4.00 (12H, m), 6.40 (1H, s), 6.50 (1H, d), 6.65 (1H, d), 7.70 (1H, d), 8.05 (1H, d);

6-Acetyl-2,3-dimethoxyphenyl-3,4-dimethoxybenzoate;

¹H-NMR (400 Mhz, DMSOd₆), ppm: 2.42 (3H, s), 3.60-4.00 (12H, four s), 7.11 (1H, d, J=9.0 Hz), 7.13 (1H, d, J=8.6 Hz), 7.55 (1H, d, J=2.0 Hz), 7.71 (1H, d, J=9.0 Hz), 7.78 (1H, dd, J=2.0;8.6 Hz);

N-{4-[3-(2-Hydroxy-3,4-dimethoxyphenyl)-3-oxopropanoyl]-2-nitrophenyl}acetamide purified by flash chromatography (eluant ethylacetate/methanol 9:1).

¹H-NMR (400 Mhz, CDCl₃), ppm: 2.38 (3H, s), 3.90 (3H, s), 3.98 (3H, s), 6.60 (1H, d), 6.78 (1H, s), 7.60 (1H, d), 8.18 (1H, dd), 8.80 (1H, ds), 9.00 (1H, d), 10.55 (1H, brs), 11.50-11.70 (2H, m);

1-(1,3-benzoxazol-5-yl)-3-(2-hydroxy-3,4-dimethoxyphenyl)-1,3-propanedione;

1-(1,3-benzothiazol-5-yl)-3-(2-hydroxy-3,4-dimethoxyphenyl)-1,3-propanedione; and

1-(6-Chloro-2-hydroxy-3,4-dimethoxyphenyl)-3-(3,4-dimethoxyphenyl)-1,3-propanedione.

### EXAMPLE 6

6-Chloro-2-(3,4-dimethoxyphenyl)-7,8-dimethoxy-4H-chromen-4-one (Compound 50)

A suspension of 1-(5-chloro-2-hydroxy-3,4-dimethoxyphenyl)-3-(3,4-dimethoxyphenyl)-1,3-propanedione (250 mg, 0.63 mmol) and sodium acetate (500 mg) in glacial acetic acid (5 mL) is refluxed for 2 hours. After cooling the precipitate is filtered, washed with acetic acid, water, ethylacetate and dried to yield 6-chloro-2-(3,4-dimethoxyphenyl)-7,8-dimethoxy-4H-chromen-4-one as white solid. Yield: 67%.

¹H-NMR (400 Mhz, DMSOd₆), ppm: 3.85 (3H, s), 3.87 (3H, s), 4.01 (3H, s), 4.08 (3H, s), 7.06 (1H, s), 7.14 (1H, d, J=8.7 Hz), 7.56 (1H, d, J=2.1 Hz), 7.67 (1H, dd, J=2.1, 8.7 Hz), 7.75 (1H, s).

By analogous procedure and starting from the appropriate 1,3-propanediones, the following compounds were prepared:

2-(2,4-Dimethoxyphenyl)-7,8-dimethoxy-4H-chromen-4-one (compound 88);

¹H-NMR (400 Mhz, DMSOd₆), ppm: 3.85-4.00 (12H, m), 6.75-6.85 (3H, m), 7.25 (1H, d), 7.70 (1H, d), 7.85 (1H, d);

N-[4-(7,8-dimethoxy-4-oxo-4H-chromen-2-yl)-2-nitrophenyl]acetamide (compound 53);

¹H-NMR (400 Mhz, DMSOd₆), ppm: 2.10 (3H, s), 3.95-3.97 (6H, m), 7.08 (1H, s), 7.30 (1H, d), 7.80 (1H, d), 7.90 (1H, d), 8.35 (1H, dd), 8.55 (1H, ds), 10.50 (1H, brs);

5-Chloro-2-(3,4-dimethoxyphenyl)-7,8-dimethoxy-4H-chromen-4-one (compound 48);

2-(1,3-benzoxazol-5-yl)-7,8-dimethoxy-4H-chromen-4-one (compound 73); and

2-(1,3-benzothiazol-5-yl)-7,8-dimethoxy-4H-chromen-4-one (compound 75).

### EXAMPLE 7

6-Chloro-2-(3,4-dihydroxyphenyl)-7,8-dihydroxy-4H-chromen-4-one (Compound 49)

A suspension of 6-chloro-2-(3,4-dimethoxyphenyl)-7,8-dimethoxy-4H-chromen-4-one (110 mg, 0.29 mmol) in a mixture of hydriodic acid (57%, 4 mL) and glacial acetic acid (4 mL) is refluxed for 15 hours. After cooling the yellow precipitate is filtered and washed with acetic acid and water. The solid is suspended into an aqueous NaHSO₃ solution and extracted with 1-butanol. The organic phase is washed with water, dried, and evaporated under reduced pressure. The crude product is washed with hot absolute ethanol and ether, dried in vacuum to yield 6-chloro-2-(3,4-dihydroxyphenyl)-7,8-dihydroxy-4H-chromen-4-one as a yellow solid. Yield: 38%.

¹H-NMR (400 Mhz, DMSOd₆), ppm: 6.61 (1H, s), 6.87 (1H, d, J=8.7 Hz), 7.43 (1H, s), 7.50-7.55 (2H, m).

By analogous procedure and starting from the appropriate chromen-4-ones, the following compounds were prepared:

2-(2,4-Dihydroxyphenyl)-7,8-dihydroxy-4H-chromen-4-one (compound 77);

obtained as mixture with 7-hydroxy-2-(2,3,4-trihydroxyphenyl)-4H-chromen-4-one.

¹H-NMR (400 Mhz, DMSOd₆), ppm: 6.40-6.50 (2H, m), 6.8043 (1H, d), 6.95 (1H, s), 7.30 (1H, d), 7.90 (1H, d); and

5-chloro-2-(3,4-dihydroxyphenyl)-7,8-dihydroxy-4H-chromen-4-one (47).

### EXAMPLE 8

1-(3,4-dimethoxyphenyl)-3-(2-hydroxy-3,4-dimethoxyphenyl)-1,3-propanedione

To 6-acetyl-2,3-dimethoxyphenyl 3,4-dimethoxybenzoate, suspended in isopropanol (100 mL) and warmed to 70° C., finely grounded potassium carbonate (7 g, 50.6 mmols) is added and the mixture is refluxed for 3.5 hours, cooled to r.t. and diluted with iced water. After acidification to pH 3 with 2N HCl, the mixture is extracted with ethyl acetate. The organic layer is separated, washed with 5% aq. sodium bicarbonate, then with brine, dried over sodium sulphate and concentrated to yield a solid that is stirred in isopropyl ether (50 mL) and filtered. Obtained 5.74 g (78% yield) of desired 1-(3,4-dimethoxyphenyl)-3-(2-hydroxy-3,4-dimethoxyphenyl)-1,3-propanedione.

¹H-NMR (400 Mhz, DMSOd₆), ppm: 3.6-4.0 (12H, m), 4.75 (2H, s), 6.7 (1H, m), 7.0 (1H, m), 7.2 (1H, s), 7.7 (1H, m), 11.0-12.0 (1H, two s).

By analogous procedure and starting from the appropriate benzoates, the following compounds were prepared:

1-(3-fluoro-4-methoxyphenyl)-3-(2-hydroxy-3,4-dimethoxyphenyl)-1,3-propanedione;

1-(4-fluoro-3-methoxyphenyl)-3-(2-hydroxy-3,4-dimethoxyphenyl)-1,3-propanedione;

1-(3-fluorophenyl)-3-(2-hydroxy-3,4-dimethoxyphenyl)-1,3-propanedione;

1-(4-fluorophenyl)-3-(2-hydroxy-3,4-dimethoxyphenyl)-1,3-propanedione; and

4-[3-(2-hydroxy-3,4-dimethoxyphenyl)-3-oxopropanoyl]benzonitrile.

### EXAMPLE 9

3-chloro-2-(3,4-dimethoxyphenyl)-7,8-dimethoxy-4H-chromen-4-one (Compound 38)

A portion of 1-(3,4-dimethoxyphenyl)-3-(2-hydroxy-3,4-dimethoxyphenyl)-1,3-propanedione (1 g, 2.77 mmol) is dissolved in dry dioxane (20 mL), sulphoryl chloride (0.25 mL, 3.1 mmol) is added and the mixture is refluxed 1 hour. The mixture is cooled to r.t. (white precipitate), iced water is added (70 mL), the precipitate is filtered, washed thoroughly with water and dried. The solid is stirred in isopropyl ether (2×40 mL) and filtered. After crystallization from dichloromethane/isopropyl ether 800 mg (76.5% yield) of white 3-chloro-2-(3,4-dimethoxyphenyl)-7,8-dimethoxy-4H-chromen-4-one is obtained.

¹H-NMR (400 Mhz, DMSOd₆), ppm: 3.8-4.0 (12H, four s), 7.18 (1H, d, J=8.6 Hz), 7.32 (1H, d, J=9 Hz), 7.5 (1H, d, J=2.0 Hz), 7.55 (1H, dd, J=2.0;8,6 Hz), 7.82 (1H, d, J=9 Hz).

### EXAMPLE 10

3-chloro-2-(3,4-dihydroxyphenyl)-7,8-dihydroxy-4H-chromen-4-one (Compound 37)

To a portion (150 mg, 0.398 mmol) of 3-chloro-2-(3,4-dimethoxyphenyl)-7,8-dimethoxy-4H-chromen-4-one, dissolved in dichloromethane (15 mL) and cooled to 0° C., a 1M solution of BBr₃ in dichloromethane (4.8 mL) is dropped slowly, the solution is stirred at rt for 2 hours, then is diluted with iced water. The pH is arranged to 6 with 5% Na₂HPO₄, the mixture is extracted with ethyl acetate, the organic layer is separated and washed with brine, dried over sodium sulphate and concentrated to yield a yellow solid that is crystallized from dichloromethane/methanol/ethyl acetate. Obtained 53 mg (42% yield) of desired 3-chloro-2-(3,4-dihydroxyphenyl)-7,8-dihydroxy-4H-chromen-4-one.

¹H-NMR (400 Mhz, DMSOd₆), ppm: 6.90 (1H, d, J=8.3 Hz), 6.97 (1H, d, J=8.7 Hz), 7.31 (1H, dd, J=2.2;8.3 Hz), 7.38 (1H, d, J=2.2 Hz), 7.42 (1H, d, J=8.7 Hz), 9.2-10.6 (4H, four s).

By analogous procedure and starting from the appropriate chromen-4-ones (42) and (40), the corresponding following compounds were prepared:

3-fluoro-2-(3,4-dihydroxyphenyl)-7,8-dihydroxy-4H-chromen-4-one (compound 41) (yield: 71%);

¹H-NMR (400 Mhz, DMSOd₆), ppm: 6.9-7.0 (2H, m), 7.4-7.5 (3H, m), 9.4-10.4 (4H, three s); and

3-cyano-2-(3,4-dihydroxyphenyl)-7,8-dihydroxy-4H-chromen-4-one (compound 39) (yield: 56%);

¹H-NMR (400 Mhz, DMSOd₆), ppm: 6.9-7.0 (2H, m), 7.41 (1H, d, J=8.7 Hz), 7.5-7.6 (2H, m), 9.4-10.8 (4H, four s).

### EXAMPLE 11

2-(3,4-dimethoxyphenyl)-3-fluoro-7,8-dimethoxy-4H-chromen-4-one (Compound 42)

To a solution of 1-(3,4-dimethoxyphenyl)-3-(2-hydroxy-3,4-dimethoxyphenyl)-1,3-propanedione (360 mg, 1 mmol) in dichloromethane (20 mL), under argon, a solution of N-fluorodibenzosulphonimide (409 mg, 1.3 mmol) in dichloromethane (20 mL) is added and the solution stirred at rt for 7 days. After solvent removal the crude product is purified by flash chromatography on silica gel, eluant: dichloromethane/methanol 100:1, obtaining 1-(3,4-dimethoxyphenyl)-2-fluoro-3-(2-hydroxy-3,4-dimethoxyphenyl)-1,3-propanedione (104 mg, 0.28 mmol, 28%).

¹H-NMR (400 Mhz, DMSOd₆), ppm: 3.6-3.9 (12H, four s), 5.7 (1H, d, J=46 Hz), 6.8 (1H, d, J=8.8 Hz), 7.04 (1H, d, J=8.5 Hz), 7.23 (1H, dd, J=2.0;8.5 Hz), 7.27 (1H, d, J=2.0 Hz), 7.52 (1H, d, J=8.8 Hz), 8.07 (1H, s).

Part of the product (19 mg, 0.05 mmol) is dissolved in glacial acetic acid (1 mL), 96% H₂SO₄ (0.01 mL) is added and the solution is refluxed for 1 hour. Water (10 mL) is added, the precipitate is filtered, washed to neutrality and dried to yield 15 mg (0.041 mmol, 82%) of desired 2-(3,4-dimethoxyphenyl)-3-fluoro-7,8-dimethoxy-4H-chromen-4-one.

¹H-NMR (400 Mhz, DMSOd₆), ppm: 3.8-4.0 (12H, four s), 7.23 (1H, d, J=8.7 Hz), 7.32 (1H, d, J=9.1 Hz), 7.51 (1H, d, J=2.0 Hz), 7.60 (1H, dd, J=2.0;8,7 Hz), 7.83 (1H, d, J=9.1 Hz).

### EXAMPLE 12

3-cyano-2-(3,4-dimethoxyphenyl)-7,8-dimethoxy-4H-chromen-4-one (Compound 40)

3-chloro-2-(3,4-dimethoxyphenyl)-7,8-dimethoxy-4H-chromen-4-one (380 mg, 1 mmol) is suspended in N-methyl pyrrolidone (5 mL), cuprous cyanide (160 mg, 1.8 mmol) is added and the mixture is refluxed overnight. The solvent is evaporated under vacuum and the crude material is purified over silica gel (eluant: dichloromethane/ethyl acetate 95:5) to yield the desired 3-cyano-2-(3,4-dimethoxyphenyl)-7,8-dimethoxy-4-oxo-4H-chromen-4-one (200 mg, 0.54 mmol, 54%).

¹H-NMR (400 Mhz, DMSOd₆), ppm: 3.9-4.0 (12H, four s), 7.3 (1H, d, J=8.6 Hz), 7.45 (1H, d, J=9 Hz), 7.65 (1H, d, J=2.0 Hz), 7.75 (1H, dd, J=2.0;8,6 Hz), 7.8 (1H, d, J=9 Hz).

### EXAMPLE 13

2-(3,4-diacetoxyphenyl)-7,8-dihydroxy-4H-chromen-4-one (Compound 90)

To a solution of 2-(3,4-dihydroxyphenyl)-7,8-dihydroxy-4H-chromen-4-one (143 mg, 0,5 mmol) in dry pyridine (4 mL) acetic anhydride (0.1 mL, 1 mmol) is added and the solution is stirred at rt overnight. Solvents are removed and the crude product is purified by flash chromatography on silica gel (eluant: dichloromethane/methanol 10:1). Obtained 2-(acetyloxy)-4-(7,8-dihydroxy-4-oxo-4H-chromen-2-yl)phenyl acetate (85 mg, 0,23 mmol, 45%) as a white solid.

¹H-NMR (400 Mhz, DMSOd₆), ppm: 2.35 (6H, two s), 6.85 (1H, s), 6.95 (1H, d, J=8.6 Hz), 7.4 (1H, d, J=8.6 Hz), 7.5 (1H, d, J=8.5 Hz), 8.05 (2H, m).

### EXAMPLE 14

8-hydroxy-7-methoxy-2-(3,4-dihydroxyphenyl)-4H-chromen-4-one (Compound 78)

To a solution of 2,3-dihydroxy-4-methoxy acetophenone (0.91 g, 5.0 mmol) in dry THF (20 mL), cooled to −78° C. under argon, lithium bis(trimethylsilyl)amide (1M solution in THF, 20 mL) is added dropwise in 15′ and the solution is stirred at −78° C. for 1 hour and at −10° C. for 2 hours. To the mixture, cooled to −78° C., a solution of methyl 3,4-di-{[tert-butyl(dimethyl)silyl]oxy}-benzoate (2.0 g, 5.0 mmol) in dry THF (10 mL) is added dropwise and the reaction mixture is stirred at −78° C. for 1 hour and at rt overnight. The mixture is poured onto ice, 20% aq. HCl (ca. 5 mL) is added (pH 2.5) and the precipitate is extracted with dichloromethane. The organic layer is separated and washed with brine, dried over sodium sulphate and concentrated to yield 1-(3,4-di-{[tert-butyl(dimethyl)silyl]oxy}-phenyl)-3-(2,3-dihydroxy-4-methoxyphenyl)-1,3-propanedione. This product is dissolved in glacial acetic acid (20 mL), 96% sulphuric acid (0.1 mL) is added and the solution is stirred at 100° C. for 1 hour, the solvent is removed under vacuum. The mixture is poured onto ice and the precipitate is extracted with ethyl acetate (×3). The organic layer is separated and washed with brine, dried over sodium sulphate and concentrated to yield a dark solid that is purified by flash chromatography on silica gel (eluant: dichloromethane/methanol 40:1). Obtained 8-hydroxy-7-methoxy-2-(3,4-dihydroxyphenyl)-4H-chromen-4-one (950 mg, 63%).

¹H-NMR (400 Mhz, DMSOd₆), ppm: 3.93 (3H, s), 6.60 (1H, s), 6.87 (1H, d, J=8.3 Hz), 7.15 (1H, d, J=9.0 Hz), 7.4-7.5 (3H, m).

By analogous procedure and starting from the appropriate ethanones and benzoates, the following compounds were obtained:

7,8-dimethoxy-2-(3,4-dihydroxyphenyl)-4H-chromen-4-one (compound 80);

2-(3,4-dihydroxyphenyl)-7-hydroxy-8-methoxy-4H-chromen-4-one (compound 92);

7-hydroxy-2-(4-hydroxy-3-methoxyphenyl)-8-methoxy-4H-chromen-4-one (compound 93);

7-hydroxy-2-(3-hydroxy-4-methoxyphenyl)-8-methoxy-4H-chromen-4-one (compound 94);

8-hydroxy-2-(4-hydroxy-3-methoxyphenyl)-7-methoxy-4H-chromen-4-one (compound 96); and

2-(3-hydroxy-4-methoxyphenyl)-7,8-dimethoxy-4H-chromen-4-one (compound 97).

### EXAMPLE 15

7,8-dihydroxy-2-(3-hydroxy-4-methoxyphenyl)-4H-chromen-4-one (Compound 79)

To a solution of methyl 3-hydroxy-4-methoxybenzoate (1.5 g, 8.2 mmol) in dry DMF (20 mL), under argon at 0° C., N-ethyldiisopropylamine (2.8 mL, 16.4 mmol) is added dropwise, followed by a solution of tert-butylchlorodimethylsilane (1.55 g, 10.3 mmol) in dry DMF (10 mL). The reaction mixture is stirred at rt overnight, iced water is added, the precipitate is filtered, washed with water and dried to yield methyl 3-{[tert-butyl(dimethyl)silyl]oxy}-4-methoxybenzoate (2.4 g, 98%) as a white powder. To a solution of 2,3,4-trihydroxy acetophenone (0.5 g, 2.97 mmol) in dry THF (20 ML), cooled to −78° C. under argon, lithium bis(trimethylsilyl)amide (1M solution in THF, 14.8 mL) is added dropwise in 15′ and the solution is stirred at −78° C. for 1 hour and at −10° C. for 2 hours. To the mixture, cooled to −78° C., a solution of methyl 3-{[tert-butyl(dimethyl)silyl]oxy}-4-methoxybenzoate (0.88 g, 2.97 mmol) in dry THF (5 mL) is added dropwise and the reaction mixture is stirred at −78° C. for 1 hour and at rt overnight. The mixture is poured onto ice, 20% aq. HCl (ca. 5 mL) is added (pH 2.5) and the precipitate is extracted with ethyl acetate. The organic layer is separated and washed with brine, dried over sodium sulphate and concentrated to yield a dark oil that is stirred in isopropyl ether/hexane (1:1) and filtered. The solid is washed with hexane and dried to 1-(3-{[tert-butyl(dimethyl)silyl]oxy}-4-methoxyphenyl)-3-(2,3,4-trihydroxyphenyl)-1,3-propanedione (0.58 g, 1.35 mmol, 45%). This product is dissolved in glacial acetic acid (10 mL), 96% sulphuric acid (0.05 mL) is added and the solution is stirred at 100° C. for 1 hour. The mixture is poured onto ice and the precipitate is extracted with ethyl acetate (×3). The organic layer is separated and washed with brine, dried over sodium sulphate and concentrated to yield a dark solid that is crystallized from dichloromethane/methanol. Obtained 7,8-dihydroxy-2-(3-hydroxy-4-methoxyphenyl)-4H-chromen-4-one (170 mg, 0.57 mmol, 42%).

¹H-NMR (400 Mhz, DMSOd₆), ppm: 3.29 (3H, s), 6.63 (1H, s), 6.91(1H, d, J=8.7 Hz), 7.07 (1H, d, J=8.6 Hz), 7.36 (1H, d, J=8.7 Hz), 7.50 (1H, d, J=2.0 Hz), 9.2-10.4 (3H, three s).

By analogous procedure and starting from the appropriate benzoate, the following compound was obtained:

7,8-dihydroxy-2-(4-hydroxy-3-methoxyphenyl)-4H-chromen-4-one (compound 91).

### EXAMPLE 16

8-hydroxy-2-(3-hydroxy-4-methoxyphenyl)-7-methoxy-4H-chromen-4-one (Compound 95) and 2-(3-hydroxy-4-methoxyphenyl)-7,8-dimethoxy-4H-chromen-4-one (Compound 100)

To a solution of 7,8-dihydroxy-2-(3,4-dihydroxyphenyl)-4H-chromen-4-one (140 mg, 0.5 mmol) in dry DMF (2 mL) anhydrous potassium carbonate (240 mg, 1.75 mmol) and methyl iodide (0.075 mL, 1.2 mmol) are added and the mixture is stirred at 55° C. overnight. The reaction mixture is purified by flash chromatography on silica gel (eluant: dichloromethane/methanol 10:1, then 4:1) and two major products are isolated: 8-hydroxy-2-(3-hydroxy-4-methoxyphenyl)-7-methoxy-4H-chromen-4-one (70 mg, 0.23 mmol, 47% yield) and 2-(3-hydroxy-4-methoxyphenyl)-7,8-dimethoxy-4H-chromen-4-one (50 mg, 0.16 mmol, 32% yield).

(compd. 95) ¹H-NMR (400 Mhz, DMSOd₆), ppm: 3.84 (3H, s), 3.93 (3H, s), 6.68 (1H, s), 7.07 (1H, d, J=8.5 Hz), 7.16 (1H, d, J=8.9 Hz), 7.44(1H, d, J=2.3 Hz), 7.5-7.6 (1H, m), 7.59 (1H, d, J=8.9 Hz).

(compd. 100) ¹H-NMR (400 Mhz, DMSOd₆), ppm: 3.85 (3H, s), 3.94 (6H, s), 6.73 (1H, s), 7.09 (1H, d, J=8.6 Hz), 7.24(1H, d, J=9.1 Hz), 7.45 (1H, d, J=2.3 Hz), 7.52 (1H, dd, J=2.3 Hz, 8.6 Hz), 7.74 (1H, d, J=9.1 Hz), 9.50 (1H, s).

### EXAMPLE 17

2-(4-Amino-3-nitrophenyl)-7,8-dimethoxy-4H-chromen-4-one (Compound 55)

A suspension of N-[4-(7,8-dimethoxy-4-oxo-4H-chromen-2-yl)-2-nitrophenyl]acetamide (200 mg, 0.52 mmol) in HCl 9N (5 mL) is refluxed for 2 hours. After cooling, the precipitate is filtered, washed with water, methanol and ether, dried in vacuum to give 2-(4-amino-3-nitrophenyl)-7,8-dimethoxy-4H-chromen-4-one as yellow solid. Yield: 92%.

¹H-NMR (400 Mhz, DMSOd₆), ppm: 3.95-3.97 (6H, m), 6.80 (1H, s), 7.15 (1H, d), 7.25 (1H, d), 7.75 (1H, d), 7.95 (1H, brs), 8.05 (1H, dd), 8.65 (1H, ds).

### EXAMPLE 18

2-(3,4-Diaminophenyl)-7,8-dimethoxy-4H-chromen-4-one (Compound 51)

2-(4-amino-3-nitrophenyl)-7,8-dimethoxy-4H-chromen-4-one (100 mg, 0.29 mmol) and NiCl₂×6H₂O (138 mg, 0.58 mmol) are suspended in a mixture methanol/DMF (4:1, 5 mL) and Zn powder (150 mg, 2.32 mmol) is added in portions with stirring. The solution is then heated at 70° C. for 2 hours. The precipitate is separated by filtration while hot, and washed with methanol. The filtrate and the washing are combined and the solvent is evaporated under reduced pressure. The crude product is then suspended in a mixture methanol/water (8:2, 10 mL), stirred for 30 min., filtered, washed with water and dried in vacuum to yield 2-(3,4-diaminophenyl)-7,8-dimethoxy-4H-chromen-4-one as yellow solid. Yield: 79%.

¹H-NMR (400 Mhz, DMSOd₆), ppm: 3.95-3.97 (6H, m), 4.80 (2H, brs), 5.35 (2H, brs), 6.45 (1H, s), 6.60 (1H, d), 7.15-7.25 (3H, m), 7.70 (1H, d).

### EXAMPLE 19

2-(3,4-Diaminophenyl)-7,8-dihydroxy-4H-chromen-4-one (Compound 52)

A suspension of 2-(3,4-diaminophenyl)-7,8-dimethoxy-4H-chromen-4-one (100 mg, 0.32 mmol) in aqueous hydrobromic acid (48%, 1 mL) is refluxed for 10 hours. After cooling, the reaction mixture is diluted with water, neutralized with 20% NaHCO₃ and extracted with 1-butanol. The organic phase is washed with water, dried, evaporated under reduced pressure and dried in vacuum to obtain 2-(3,4-diaminophenyl)-7,8-dihydroxy-4H-chromen-4-one as yellow solid. Yield: 40%.

¹H-NMR (400 Mhz, DMSOd₆), ppm: 5.60-6.00 (4H, m), 6.40 (1H, s), 6.65 (1H, d), 6.90 (1H, d), 7.30-7.40 (3H, m), 9.20 (1H, brs), 10.20 (1H, brs).

By analogous procedure and starting from compound 55 the following compound was obtained:

2-(4-amino-3-nitrophenyl)-7,8-dihydroxy-4H-chromen-4-one (compound 56).

### EXAMPLE 20

5-(7,8-Dimethoxy-4-oxo-4H-chromen-2-yl)-1,3-dihydro-2H-benzimidazol-2-one (Compound 67)

A suspension of 2-(3,4-diaminophenyl)-7,8-dimethoxy-4H-chromen-4-one (100 mg, 0.32 mmol) in dry tetrahydrofuran (5 mL) is cooled (0° C.) and N,N′-carbonyldiimidazole (62 mg, 0.38 mmol) is added rapidly with stirring. The cooling bath is removed and stirring is continued overnight. The reaction mixture is then filtered and the solid is washed with tetrahydrofuran, methanol and ether, dried in vacuum to yield 5-(7,8-dimethoxy-4-oxo-4H-chromen-2-yl)-1,3-dihydro-2H-benzimidazol-2-one as yellow solid. Yield: 80%.

¹H-NMR (400 Mhz, DMSOd₆), ppm: 3.95-3.98 (6H, m), 6.80 (1H, s), 7.10 (1H, d), 7.25 (1H, d), 7.55 (1H, ds), 7.70 (1H, dd), 7.80 (1H, d), 10.90 (1H, brs), 11.0 (1H, brs).

By analogous procedure starting from the diamino derivative 51 and by using appropriate condensing agents, such as N,N′-thiocarbonyldiimidazole, cyanogen bromide, and chloroacetyl cloride, the corresponding compounds below were obtained:

7,8-dimethoxy-2-(2-sulfanyl-1H-benzimidazol-5-yl)-4H-chromen-4-one (compound 69);

2-(2-amino-1H-benzimidazol-5-yl)-7,8-dimethoxy-4H-chromen-4-one (compound 71);

6-(7,8-dimethoxy-4-oxo-4H-chromen-2-yl)-3,4-dihydro-2(1H)-quinoxalinone (compound 83); and

7-(7,8-dimethoxy-4-oxo-4H-chromen-2-yl)-3,4-dihydro-2(1H)-quinoxalinone (compound 85).

### EXAMPLE 21

2-(1H-1,2,3-Benzotriazol-5-yl)-7,8-dimethoxy-4H-chromen-4-one (Compound 65)

To a solution of sodium nitrite (50 mg, 0.70 mmol) in water (1 mL) cooled at 0° C., a suspension of 2-(3,4-diaminophenyl)-7,8-dimethoxy-4H-chromen-4-one (200 mg, 0.64 mmol) in water/glacial acetic acid (2:1, 3 mL) is added maintaining the temperature below 5° C. The reaction mixture is then stirred at 15° C. for 3 hours, filtered, washing the solid with water. The crude product is boiled with methanol (15 mL) for 30 min., filtered while hot, washed with methanol and ether, dried in vacuum to obtain 2-(1H-1,2,3-benzotriazol-5-yl)-7,8-dimethoxy-4H-chromen-4-one as grey solid. Yield: 43%.

¹H-NMR (400 Mhz, DMSOd₆), ppm: 3.95 (3H, s), 3.98 (3H, s), 7.10 (1H, s), 7.30 (1H, d), 7.80 (1H, d), 8.05-8.20 (2H, m), 8.60 (1H, s), 12.0 (1H, brs).

### EXAMPLE 22

N-[2-(acetylamino)-4-(7,8-dimethoxy-4-oxo-4H-chromen-2-yl)phenyl]acetamide (Compound 58)

A suspension of 2-(3,4-diaminophenyl)-7,8-dimethoxy-4H-chromen-4-one (150 mg, 0.48 mmol) and trietylamine (0.53 mL, 3.84 mmol) in dry tetrahydrofuran (3 mL) is cooled (0° C.) and acetylchloride (0.14 mL, 1.92 mmol) is added with stirring. The cooling bath is removed and stirring is continued overnight. The reaction mixture is then filtered and the solid is washed with tetrahydrofuran and dried. The crude product is suspended in water (15 mL) stirred at room temperature for 30 min., filtered, washed with water and dried in vacuum at 50° C. to yield N-[2-(acetylamino)-4-(7,8-dimethoxy-4-oxo-4H-chromen-2-yl)phenyl]acetamide as yellow solid. Yield: 33%.

¹H-NMR (400 Mhz, DMSOd₆), ppm: 3.95-3.98 (6H, m), 6.80 (1H, s), 7.25 (1H, d), 7.75 (1H, ds), 7.80-7.90 (2H, m), 8.30 (1H, s), 9.50-9.52 (2H, m).

By analogous procedure and starting from compounds (56) and (52) the corresponding following compounds were obtained:

2-(4-acetylamino-3-nitrophenyl)-7,8-dihydroxy-4H-chromen-4-one (compound 54); and

2-(3,4-diacetylaminophenyl)-7,8-dihydroxy-4H-chromen-4-one (compound 57).

By analogous procedure by using the appropriate acylating agent, such as trifluoroacetic anhydride, and starting from compounds (51) and (52), the corresponding following compounds were obtained:

2-(3,4-di-trifluoroacetylaminophenyl)-7,8-dimethoxy-4H-chromen-4-one (compound 60); and

2-(3,4-di-trifluoroacetylaminophenyl)-7,8-dihydroxy-4H-chromen-4-one (compound 59).

### EXAMPLE 23

2-(1H-Benzimidazol-5-yl)-7,8-dimethoxy-4H-chromen-4-one (Compound 61)

A suspension of 2-(3,4-diaminophenyl)-7,8-dimethoxy-4H-chromen-4-one (200 mg, 0.64 mmol) in a mixture of 4N hydrochloric acid (5 mL) and formic acid (1 mL) is heated at 100° C. for 2 hours. After cooling the reaction mixture is carefully neutralized with sodium bicarbonate powder and the solid precipitated is filtered, washed with water, methanol and ether, dried in vacuum to obtain 2-(1H-benzimidazol-5-yl)-7,8-dimethoxy-4H-chromen-4-one as white solid. Yield: 77%.

¹H-NMR (400 Mhz, DMSOd₆), ppm: 3.96 (3H, s), 3.98 (3H, s), 6.95 (1H, s), 7.25 (1H, d), 7.70-8.00 (4H, m), 8.40 (1H, s), 11.90 (1H, brs)

### EXAMPLE 24

7,8-dimethoxy-2-(3,4-dihydroxyphenyl)-4H-chromen-4-one (Compound 80)

To a solution of methyl 3,4-dihydroxy-benzoate (0.84 g, 5 mmol) in dry DMF (40 mL), under argon at 0° C., N-ethyldiisopropylamine (3.4 mL, 20 mmol) is added dropwise, followed by a solution of tert-butylchlorodimethylsilane (0.85 g, 11 mmol) in dry DMF (6 mL). The reaction mixture is stirred at rt overnight, iced water is added, the precipitate is filtered, washed with water and dried to yield methyl 3,4-di-{[tert-butyl(dimethyl)silyl]oxy}-benzoate (1.9 g, 96%) as a white powder.

To a solution of 2-hydroxy-3,4-dimethoxy acetophenone (0.85 g, 4.35 mmol) in dry THF (20 mL), cooled to −78° C. under argon, lithium bis(trimethylsilyl)amide (1M solution in THF, 13 ml) is added dropwise in 15′ and the solution is stirred at −78° C. for 1 hour and at −10° C. for 2 hours. To the mixture, cooled to −78° C., a solution of methyl 3,4-di-{[tert-butyl(dimethyl)silyl]oxy}-benzoate (1.72 g, 4.35 mmol) in dry THF (10 mL) is added dropwise and the reaction mixture is stirred at −78° C. for 1 hour and at rt overnight. The mixture is poured onto ice, 20% aq. HCl (ca. 4 mL) is added (pH 2.5) and the precipitate is extracted with ethyl acetate. The organic layer is separated and washed with brine, dried over sodium sulphate and concentrated to yield a dark oil that is stirred in isopropyl ether/hexane (1:1) and filtered. The solid is washed with hexane and dried overnight to yield 1.95 g (80%) 1-(3,4-di-{[tert-butyl(dimethyl)silyl]oxy}-phenyl)-3-(2-hydroxy-3,4-dimethoxyphenyl)-1,3-propanedione. This product is dissolved in glacial acetic acid (20 mL), 96% sulphuric acid (0.1 ml) is added and the solution is stirred at 100° C. for 1 hour, the solvent is removed under vacuum. The mixture is poured onto ice and the precipitate is extracted with ethyl acetate (×3). The organic layer is separated and washed with brine, dried over sodium sulphate and concentrated to yield a dark solid that is crystallized from dichloromethane/methanol. Obtained 7,8-dimethoxy-2-(3,4-dihydroxyphenyl)-4H-chromen-4-one (940 mg, 3 mmol, 86%).

¹H-NMR (400 Mhz, DMSOd₆), ppm: 3.94 (6H, s), 6.65 (1H, s), 6.89 (1H, d, J=8.3 Hz), 7.23 (1H, d, J=9.0 Hz), 7.39 (1H, dd, J=8.3 Hz,2.2 Hz), 7.43 (1H, d, J=2.2 Hz), 7.73 (1H, d, J=9.0 Hz), 9.4 (1H, s), 9.8 (1H, s).

### EXAMPLE 25

2-(1H-Benzimidazol-5-yl)-7,8-dihydroxy-4H-chromen-4-one and 2-(1H-benzimidazol-5-yl)-8-hydroxy-7-methoxy-4H-chromen-4-one (Compounds 62 and 63)

A suspension of 2-(1H-benzimidazol-5-yl)-7,8-dimethoxy-4H-chromen-4-one (200 mg, 0.62 mmol) in 48% aqueous hydrobromic acid (2 mL) is heated at reflux for 6 hours. After cooling the reaction mixture is carefully neutralized with sodium bicarbonate powder and the solid precipitated is filtered, washed with water, methanol and ether, dried in vacuum to obtain a mixture of 62 and 63 in about 1:1 ratio. The mixture is then separated by preparative HPLC to give 2-(1H-benzimidazol-5-yl)-7,8-dihydroxy-4H-chromen-4-one as white solid (50 mg, 27%) and 2-(1H-benzimidazol-5-yl)-8-hydroxy-7-methoxy-4H-chromen-4-one as yellowish solid (60 mg, 31%).

2-(1H-Benzimidazol-5-yl)-7,8-dihydroxy-4H-chromen-4-one ¹H-NMR (400 Mhz, DMSOd₆), ppm: 6.88-6.92 (2H, m), 7.40 (1H, d), 7.80 (1H, d), 8.10 (1H, d), 8.50 (1H, s), 8.80 (1H, brs); and

2-(1H-Benzimidazol-5-yl)-8-hydroxy-7-methoxy-4H-chromen-4-one ¹H-NMR (400 Mhz, DMSOd₆), ppm: 3.98 (3H, s), 7.00 (1H, s), 7.20 (1H, d), 7.50 (1H, d), 7.82 (1H, d), 8.05 (1H, d), 8.45 (1H, s), 8.82 (1H, brs).

By analogous procedure and starting from the appropriate chromen-4-ones, the following compounds were prepared:

5-(7,8-Dihydroxy-4-oxo-4H-chromen-2-yl)-1,3-dihydro-2H-benzimidazol-2-one (compound 68); Yield: 60%.

¹H-NMR (400 Mhz, DMSOd₆), ppm: 6.70 (1H, s), 6.95 (1H, d), 7.05 (1H, d), 7.38 (1H, d), 7.65 (1H, s), 7.75 (1H, d), 9.40 (1H, s), 10.25 (1H, s), 10.90 (1H, brs), 10.95 (1H, brs);

7,8-Dihydroxy-2-(2-methyl-1H-benzimidazol-5-yl)-4H-chromen-4-one (compound 102); Yield: 62%.

¹H-NMR (400 Mhz, DMSOd₆), ppm: 2.55 (3H, s), 6.80 (1H, s), 6.95 (1H, d), 7.40 (1H, d), 7.60 (1H, d), 7.90 (1H, d), 8.30 (1H, s), 9.40 (1H, brs), 10.20 (1H, brs);

7,8-dihydroxy-2-(2-sulfanyl-1H-benzimidazol-5-yl)-4H-chromen-4-one (compound 70);

2-(2-amino-1H-benzimidazol-5-yl)-7,8-dihydroxy-4H-chromen-4-one (compound 72);

6-(7,8-dihydroxy-4-oxo-4H-chromen-2-yl)-1,4-dihydro-2,3-quinoxalinedione (compound 82);

6-(7,8-dihydroxy-4-oxo-4H-chromen-2-yl)-3,4-dihydro-2(1H)-quinoxalinone (compound 84);

7-(7,8-dihydroxy-4-oxo-4H-chromen-2-yl)-3,4-dihydro-2(1H)-quinoxalinone (compound 86);

2-(1,3-benzoxazol-5-yl)-7,8-dihydroxy-4H-chromen-4-one (compound 74);

2-(1,3-benzothiazol-5-yl)-7,8-dihydroxy-4H-chromen-4-one (compound 76); and

2-(1H-1,2,3-benzotriazol-5-yl)-7,8-dihydroxy-4H-chromen-4-one (compound 66).

### EXAMPLE 26

7,8-Dimethoxy-2-(2-methyl-1H-benzimidazol-5-yl)-4H-chromen-4-one (Compound 101)

A suspension of 2-(3,4-diaminophenyl)-7,8-dimethoxy-4H-chromen-4-one (500 mg, 1.60 mmol) in a mixture of 4N hydrochloric acid (5 mL) and glacial acetic acid (2.5 mL) is heated at 100° C. for 2 hours. After cooling the reaction mixture is carefully neutralized with sodium bicarbonate powder and the solid precipitated is extracted with chloroform (3×50 mL). The organic phase is dried with anhydrous sodium sulfate, filtered and evaporated. The crude product is purified using boiling methanol to obtain pure 7,8-dimethoxy-2-(2-methyl-1H-benzimidazol-5-yl)-4H-chromen-4-one as white solid. Yield: 43%.

¹H-NMR (400 Mhz, DMSOd₆), ppm: 2.55 (3H, s), 3.95-4.00 (6H, m), 6.80 (1H, s), 6.95 (1H, s), 7.25 (1H, d), 7.60 (1H, brs), 7.80 (1H, d), 7.85 (1H, d), 8.20 (1H, brs).

### EXAMPLE 27

6-(7,8-Dimethoxy-4-oxo-4H-chromen-2-yl)-1,4-dihydro-2,3-quinoxalinedione (Compound 81)

To a solution of 2-(3,4-diaminophenyl)-7,8-dimethoxy-4H-chromen-4-one (100 mg, 0.32 mmol) in anhydrous DMF (1 mL), at room temperature, 1,1′-oxalyl-diimidazol (91 mg, 0.48 mmol) is added. and the mixture is stirred for 24 hours. Methanol (5 mL) is added and the suspension is stirred at 50° C. for 30 min. After cooling the yellow solid precipitated is filtered, washed with methanol, ether and dried in vacuum to obtain 6-(7,8-dimethoxy-4-oxo-4H-chromen-2-yl)-1,4-dihydro-2,4-quinoxalinedione as yellowish solid. Yield: 51%.

¹H-NMR (400 Mhz, DMSOd₆), ppm: 2.55 (3H, s), 3.90-4.00 (6H, m), 6.80 (1H, s), 7.20-7.30 (2H, m), 7.70-7.85 (3H, m)

### EXAMPLE 28

2-{2-[3-(Dimethylamino)propyl]-1H-benzimidazol-5-yl}-7,8-dimethoxy-4H-chromen-4-one (Compound 103)

A suspension of 2-(3,4-diaminophenyl)-7,8-dimethoxy-4H-chromen-4-one (200 mg, 0.64 mmol) and 4-(dimethylamino)butyric acid hydrochloride (3.2 g, 19.2 mmol) in 4N hydrochloric acid (10 mL) is heated at reflux for 48 hours. After cooling to the mixture sodium bicarbonate powder is carefully added until basic condition and the solid precipitated is extracted with dichloromethane (3×20 mL). The organic phase is dried with anhydrous sodium sulfate, filtered and evaporated. The crude product is purified by flash chromatography (eluant dichloromethane/methanol in different ratios, 9:1, 8:2, 7:3 and 1:1) to yield 2-{2-[3-(dimethylamino)propyl]-1H-benzimidazol-5-yl}-7,8-dimethoxy-4H-chromen-4-one as yellowish solid. Yield 35%.

¹H-NMR (400 Mhz, CDCl₃), ppm: 2.00-2.10 (2H, m), 2.45 (6H, s), 2.60 (2H, t), 3.20 (2H, t), 4.00 (3H, s), 4.08 (3H, s), 6.80 (1H, s), 7.05 (1H, d), 7.60 (1H, d), 7.80 (1H, d), 7.95 (1H, d), 8.20 (1H, s).

### EXAMPLE 29

Intramuscular Injection of 50 mg/ml

A pharmaceutical injectable composition can be manufactured by dissolving 50 g of 3-cyano-2-(3,4-dihydroxyphenyl)-7,8-dihydroxy-4H-chromen-4-one (compound 39) in sterile propylene glycol (1000 ml) and sealed in 1-5 ml ampoules.

The present disclosure is an exemplification of the principles of the invention and is not intended to limit the invention to the particular embodiments illustrated. Those skilled in the art may recognize other equivalents to the specific embodiment described herein which equivalents are intended to be encompassed by the claims attached hereto.

What is claimed is:

1. A method for inhibiting telomerase enzyme, which comprises contacting said enzyme with an effective amount of a compound having the following formula (I) 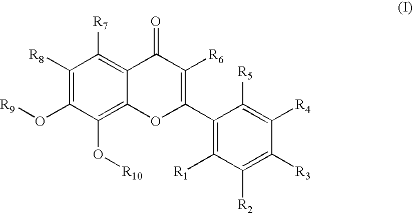
wherein
each of R₁, R₂ and R₅ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino, C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl;
each of R₃ and R₄ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino, C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl, or R₃ and R₄, taken together, represent a 5 or 6 membered fused ring system having the following formula
—Y—(y)—X—(x)—Z—
wherein
(iii) when X represents methylene, —(y)— represents a single bond, —(x)— represents a single bond, Y and Z both represent oxygen (O) or sulphur (S);
(iv) when X represents CR, —(y)— represents a double bond, —(x)— represents a single bond, Y represents N, Z represents NR′, oxygen (O) or sulphur (S), R represents hydrogen or C₁-C₆ alky optionally substituted with C₁-C₆ dialkylamino and R′ represents hydrogen, C₁-C₆ alkyl, or C₁-C₆ acyl;
(iii) when X represents C—OR, —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;
(iv) when X represents C═O, —(y)— represents a single bond, —(x)— represents a single bond, Y represents NR′, Z represents NR′, oxygen (O) or sulphur (S) and R′ represents hydrogen, C₁-C₆ alkyl, or C₁-C₆ acyl;
(v) when X represents C—SR, —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;
(vi) when X represents C═S, —(y)— represents a single bond, —(x)— represents a single bond, Y represents NR′, Z represents NR′, oxygen (O) or sulphur (S) and R′ represents hydrogen, C₁-C₆ alkyl or C₁-C₆ acyl;
(vii) when X represents N, —(y)— represents a double bond, —(x)— represents a single bond, Y represents N, Z represents NR′ and R′ represents hydrogen, C₁-C₆ alkyl or C₁-C₆ acyl;
(viii) when X represents O═C—C═O, —(y)— represents a single bond, —(x)— represents a single bond, Y and Z represent NR′ wherein R′ represents hydrogen, C₁-C₆ alkyl or C₁-C₆ acyl;
(ix) when X represents RO—C—C═O, —(y)— represents a double bond, —(x)— represents a single bond, Y represents N, Z represents NR′ and each of R and R′ represents, independently, hydrogen, C₁-C₆ alkyl or C₁-C₆ acyl;
(x) when X represents RO—C—C—OR, —(y)— represents a double bond, —(x)— represents a double bond, Y and Z are N, where R represents hydrogen, C₁-C₆ alkyl or C₁-C₆ acyl;
(xi) when X represents CH₂—CO, —(y)— represents a single bond, —(x)— represents a single bond, Y represents NR, Z represents NR′ and each of R and R′ represents, independently, hydrogen, C₁-C₆ alkyl or C₁-C₆ acyl;
(xii) when X represents CH₂—C—OR′, —(y)— represents a single bond, —(x)— represents a double bond, Y represents NR, Z represents N and each of R and R′ represents, independently, hydrogen, C₁-C₆ alkyl or C₁-C₆ acyl;
(xiii) when X represents CO—CH₂, —(y)— represents a single bond, —(x)— represents a single bond, Y represents NR′, Z represents NR and each of R and R′ represents, independently, hydrogen, C₁-C₆ alkyl or C₁-C₆ acyl; or
(xiv) when X represents R′O—C—CH₂, —(y)— represents a double bond, —(x)— represents a single bond, Y represents N, Z represents NR and each of R and R′ represents, independently, hydrogen, C₁-C₆ alkyl or C₁-C₆ acyl;
R₆ represents hydrogen, halogen, cyano, NRₐR_{b} in which each of Rₐ and R_{b} represents, independently, hydrogen, C₁-C₆ alkyl, C₁-C₄ acyl, aroyl, C₁-C₆ alkylsulfonyl or arylsulfonyl;
each of R₇ and R₈ represents, independently, hydrogen, halogen, cyano, C₁-C₆ alkoxy, C₁-C₆ acyloxy, aroyloxy, hydroxy, C₁-C₆ trialkylsilyloxy, aryl C₁-C₆ dialkylsilyloxy, alkyl C₁-C₆ diarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl;
each of R₉ and R₁₀ represents, independently, hydrogen; C₁-C₆ alkyl unsubstituted or substituted by aryl; C₁-C₆ acyl; aroyl; C₁-C₆ trialkylsilyl; aryl C₁-C₆ dialkylsilyl; C₁-C₆ alkyldiarylsilyl; triarylsilyl; C₁-C₆ alkoxycarbonyl; or R₉ and R₁₀, taken together, represent methylene or carbonyl;
or a pharmaceutically acceptable salts thereof.

2. A method for treating a telomerase-modulated disease, which comprises administering to a mammal a therapeutic effective amount of a compound of formula (I) as defined in claim 1 or a pharmaceutically acceptable salt thereof.

3. A method for treating a cancer disease related to a deranged cancer cell growth mediated by telomerase enzyme activity, which comprises administering to a mammal a therapeutic effective amount of a compound of formula (I) as defined in claim 1 or a pharmaceutically acceptable salt thereof.

4. A method for treating a cancer, which comprises administering to a mammal a therapeutic effective amount of a compound of formula (I) as defined in claim 1 or a pharmaceutically acceptable salt thereof.

5. A compound of formula (I) as defined in claim 1, for use in the preparation of a medicament having anticancer activity.

6. A pharmaceutical formulation for treating a telomerase-modulated disease, which comprises a compound of formula (I) as defined in claim 1 or a pharmaceutically acceptable-salt thereof and a pharmaceutically acceptable excipient.

7. A pharmaceutical formulation for treating a cancer disease related to a deranged cancer cell growth mediated by telomerase enzyme activity, which comprises a compound of formula (I) as defined in claim 1 or a pharmaceutically acceptable salt thereof and a pharmaceutically acceptable excipient.

8. A pharmaceutical formulation for treating a cancer, which comprises a compound of formula (I) as defined in claim 1 or a pharmaceutically acceptable salt thereof and a pharmaceutically acceptable excipient.

9. A compound of formula (Ia) 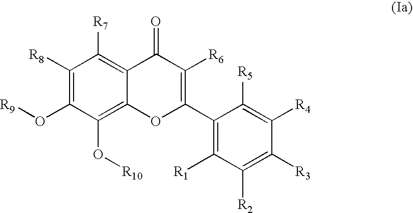
wherein
R₆ represents halogen, cyano, NRₐR_{b} in which each of Rₐ and R_{b} represents, independently, hydrogen, C₁-C₆ alkyl, C₁-C₄ acyl, aroyl, C₁-C₆ alkylsulfonyl or arylsulfonyl;
each of R₃, R₄ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl, or
R₃ and R₄, taken together, represent a 5 or 6 membered fused ring system having the following formula
—Y—(y)—X—(x)—Z—
wherein
(iv) when X represents methylene, —(y)— represents a single bond, —(x)— represents a single bond, Y and Z both represent oxygen (O) or sulphur (S);
(v) when X represents CR, —(y)—0 represents a double bond, —(x)—0represents a single bond, Y represents N, Z represents NR′, oxygen (O) or sulphur (S), R represents hydrogen or C₁-C₆ alky optionally substituted with C₁-C₆ dialkylamino and R′ represents hydrogen, C₁-C₆ alkyl, or C₁-C₆ acyl;
(iii) when X represents C—OR , —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;
(iv) when X represents C═O, —(y)— represents a single bond, —(x)— represents a single bond, Y represents NR′, Z represents NR′, oxygen (O) or sulphur (S) and R′ represents hydrogen, C₁-C₆ alkyl, or C₁-C₆ acyl;
(v) when X represents C—SR, —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;
(vi) when X represents C═S, —(y)— represents a single bond, —(x)— represents a single bond, Y represents NR′, Z represents NR′, oxygen (O) or sulphur (S) and R′ represents hydrogen, C₁-C₆ alkyl or C₁-C₆ acyl;
(vii) when X represents N, —(y)— represents a double bond, —(x)— represents a single bond, Y represents N, Z represents NR′ and R′ represents hydrogen, C₁-C₆ alkyl or C₁-C₆ acyl;
(viii) when X represents O═C—C═O, —(y)— represents a single bond, —(x)— represents a single bond, Y and Z represent NR′ wherein R′ represents hydrogen, C₁-C₆ alkyl or C₁-C₆ acyl;
(ix) when X represents RO—C—C═O, —(y)— represents a double bond, —(x)— represents a single bond, Y represents N, Z represents NR′ and each of R and R′ represents, independently, hydrogen, C₁-C₆ alkyl or C₁-C₆ acyl;
(x) when X represents RO—C—C—OR, —(y)— represents a double bond, —(x)— represents a double bond, Y and Z are N, where R represents hydrogen, C₁-C₆ alkyl or C₁-C₆ acyl;
(xi) when X represents CH₂—CO, —(y)— represents a single bond, —(x)— represents a single bond, Y represents NR, Z represents NR′ and each of R and R′ represents, independently, hydrogen, C₁-C₆ alkyl or C₁-C₆ acyl;
(xii) when X represents CH₂—C—OR′, —(y)— represents a single bond, —(x)— represents a double bond, Y represents NR, Z represents N and each of R and R′ represents, independently, hydrogen, C₁-C₆ alkyl or C₁-C₆ acyl;
(xiii) when X represents CO—CH₂, —(y)— represents a single bond, —(x)— represents a single bond, Y represents NR′, Z represents NR and each of R and R′ represents, independently, hydrogen, C₁-C₆ alkyl or C₁-C₆ acyl; or
(xiv) when X represents R′O—C—CH₂, —(y)— represents a double bond, —(x)— represents a single bond, Y represents N, Z represents NR and each of R and R′ represents, independently, hydrogen, C₁-C₆ alkyl or C₁-C₆ acyl;
each of R₁, R₂ and R₅ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl, or arylaminosulfonyl;
each of R₇ and R₈ represents, independently, hydrogen, halogen, cyano, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfony or arylaminosulfonyl;
each of R₉ and R₁₀ represents, independently, hydrogen, C₁-C₆ alkyl unsubstituted or substituted by aryl; C₁-C₆ acyl; aroyl; C₁-C₆ trialkylsilyl; aryl C₁-C₆ dialkylsilyl; C₁-C₆ alkyldiarylsilyl; triarylsilyl, C₁-C₆ alkoxycarbonyl; or R₉ and R₁₀, taken together, represents methylene or carbonyl;
or a pharmaceutically acceptable salt thereof.

10. A compound of formula (Ib) 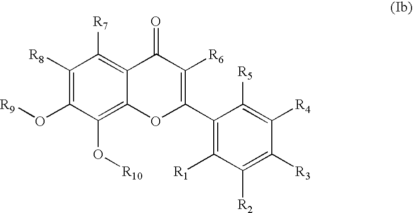
wherein:
R₆ is hydrogen;
R₃ and R₄, taken together, represent a 5 or 6 membered fused ring system having the following formula
—Y—(y)—X—(x)—Z—
wherein
(ii) when X represents CR, —(y)— represents a double bond, —(x)— represents a single bond, Y represents N, Z represents NR′, oxygen (O) or sulphur (S), R represents hydrogen or C₁-C₆ alky optionally substituted with C₁-C₆ dialkylamino and R′ represents hydrogen, C₁-C₆ alkyl, or C₁-C₆ acyl;
(iii) when X represents C—OR , —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;
(iv) when X represents C═O, —(y)— represents a single bond, —(x)— represents a single bond, Y represents NR′, Z represents NR′, oxygen (O) or sulphur (S) and R′ represents hydrogen, C₁-C₆ alkyl, or C₁-C₆ acyl;
(v) when X represents C—SR, —(y)— represents a double bond, —(x)— represents a single bond, Y represents N, Z represents NR′, oxygen (O) or sulphur (S) and each of R and R′ represents, independently, hydrogen, C₁-C₆ alkyl or C₁-C₆ acyl;
(vi) when X represents C═S, —(y)— represents a single bond, —(x)—0 represents a single bond, Y represents NR′, Z represents NR′, oxygen (O) or sulphur (S) and R′ represents hydrogen, C₁-C₆ alkyl or C₁-C₆ acyl;
(vii) when X represents N, —(y)— represents a double bond, —(x)—0 represents a single bond, Y represents N, Z represents NR′ and R′ represents hydrogen, C₁-C₆ alkyl or C₁-C₆ acyl;
(viii) when X represents O═C—C═O, —(y)— represents a single bond, —(x)— represents a single bond, Y and Z represent NR′ wherein R′ represents hydrogen, C₁-C₆ alkyl or C₁-C₆ acyl;
(ix) when X represents RO—C—C═O, —(y)— represents a double bond, —(x)— represents a single bond, Y represents N, Z represents NR′ and each of R and R′ represents, independently, hydrogen, C₁-C₆ alkyl or C₁-C₆ acyl;
(x) when X represents RO—C—C—OR, —(y)— represents a double bond, —(x)— represents a double bond, Y and Z are N, where R represents hydrogen, C₁-C₆ alkyl or C₁-C₆ acyl;
(xi) when X represents CH₂—CO, —(y)— represents a single bond, —(x)—0 represents a single bond, Y represents NR, Z represents NR′ and each of R and R′ represents, independently, hydrogen, C₁-C₆ alkyl or C₁-C₆ acyl;
(xii) when X represents CH₂—C—OR′, —(y)— represents a single bond, —(x)— represents a double bond, Y represents NR, Z represents N and each of R and R′ represents, independently, hydrogen, C₁-C₆ alkyl or C₁-C₆ acyl;
(xiii) when X represents CO—CH₂, —(y)— represents a single bond, —(x)— represents a single bond, Y represents NR′, Z represents NR and each of R and R′ represents, independently, hydrogen, C₁-C₆ alkyl or C₁-C₆ acyl; or
(xv) when X represents R′O—C—CH₂, —(y)— represents a double bond, —(x)— represents a single bond, Y represents N, Z represents NR and each of R and R′ represents, independently, hydrogen, C₁-C₆ alkyl or C₁-C₆ acyl;
each of R₁, R₂ and R₅ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl;
each of R₇ and R₈ represents, independently, hydrogen, halogen, cyano, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl, or arylaminosulfonyl;
each of R₉ and R₁₀ represents, independently, hydrogen; C₁-C₆ alkyl unsubstituted or substituted by an aryl; C₁-C₆ acyl; aroyl; C₁-C₆ trialkylsilyl; aryl C₁-C₆ dialkylsilyl; C₁-C₆ alkyldiarylsilyl; triarylsilyl; C₁-C₆ alkoxycarbonyl; or R₉ and R₁₀, taken together, represent methylene or carbonyl;
or a pharmaceutically acceptable salt thereof.

11. A compound of formula (Ic) 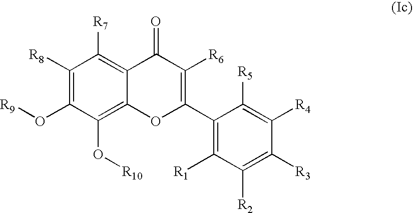
wherein
R₆ is hydrogen;
each of R₃ and R₄ represents, independently, hydrogen, halogen, cyano, hydroxy, C₁-C₆ alkoxy, C₁-C₆ acyloxy, aroyloxy, C₁-C₆ trialkylsilyloxy, arylC₁-C₆di alkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino arylsulfonylamino, C₁-C₆ alkylaminosulfonyl, and arylaminosulfonyl; each of R₁, R₂ and R₅ represents, independently, hydrogen, halogen, cyano, hydroxy, C₁-C₆ alkoxy, C₁-C₆ acyloxy, aroyloxy, C₁-C₆ trialkylsilyloxy, aryldi C₁-C₆ alkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆alkylsulfonylamino, arylsulfonylamino, C₁-C₆ alkylaminosulfonyl or arylaminosulfonyl;
each of R₇ and R₈ represents, independently, halogen, cyano, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl, or arylaminosulfonyl, provided that R₇ and R₈ are not contemporarily hydrogen;
each of R₉ and R₁₀ represents, independently, hydrogen, C₁-C₆ alkyl unsubstituted or substituted by an aryl, C₁-C₆ acyl, aroyl, C₁-C₆ trialkylsilyl, aryl C₁-C₆ di alkylsilyl, C₁-C₆ alkyldiarylsilyl, triarylsilyl or C₁-C₆ alkoxycarbonyl, or R₉ and R₁₀, taken together, represent methylene or a carbonyl;
or a pharmaceutically acceptable salt thereof.

12. A compound of formula (Ia) according to claim 9 wherein
R₆ is as defined in claim 9;
each of R₃ and R₄ represents, independently, hydrogen, halogen, cyano, hydroxy, C₁-C₆ alkoxy, C₁-C₆ acyloxy, aroyloxy, C₁-C₆ trialkylsilyloxy, aryl C₁-C₆ dialkylsilyloxy, C₁-C₆ alkyldiarylsilyloxy, triarylsilyloxy, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl or arylaminosulfonyl, or
R₃ and R₄, taken together, represent a 5 or 6 membered fused ring system having the following formula
—Y—(y)—X—(x)—Z—
wherein
(i) when X represents methylene, —(y)— represents a single bond, —(x)— represents a single bond, Y and Z both represent oxygen (O) or sulphur (S);
(vi) when X represents CR, —(y)— represents a double bond, —(x)— represents a single bond, Y represents N, Z represents NR′, oxygen (O) or sulphur (S), R represents hydrogen or C₁-C₆ alky optionally substituted with C₁-C₆ dialkylamino and R′ represents hydrogen or C₁-C₆ alkyl;
(iii) when X represents C—OR, —(y)— represents a double bond, —(x)— represents a single bond, Y represents N, Z represents NR′, oxygen (O) or sulphur (S) and each of R and R′ represents, independently, hydrogen or C₁-C₆ alkyl;
(iv) when X represents C═O, —(y)— represents a single bond, —(x)— represents a single bond, Y represents NR′, Z represents NR′, oxygen (O) or sulphur (S) and R′ represents hydrogen or C₁-C₆ alkyl;
(v) when X represents C—SR, —(y)— represents a double bond, —(x)— represents a single bond, Y represents N, Z represents NR′, oxygen (O) or sulphur (S) and each of R and R′ represents, independently, hydrogen or C₁-C₆ alkyl;
(vi) when X represents C═S, —(y)— represents a single bond, —(x)— represents a single bond, Y represents NR′, Z represents NR′, oxygen (O) or sulphur (S) and R′ represents hydrogen or C₁-C₆ alkyl;
(vii) when X represents N, —(y)— represents a double bond, —(x)— represents a single bond, Y represents N, Z represents NR′ and R′ represents hydrogen or C₁-C₆ alkyl;
(viii) when X represents O═C—C═O, —(y)— represents a single bond, —(x)— represents a single bond, Y and Z represent NR′ wherein R′ represents hydrogen or C₁-C₆ alkyl;
(ix) when X represents RO—C—C═O, —(y)— represents a double bond, —(x)— represents a single bond, Y represents N, Z represents NR′ and each of R and R′ represents, independently, hydrogen or C₁-C₆ alkyl;
(x) when X represents RO—C—C—OR, —(y)— represents a double bond, —(x)— represents a double bond, Y and Z represent N and R represents hydrogen or C₁-C₆ alkyl;
(xi) when X represents CH₂—CO, —(y)— represents a single bond, —(x)— represents a single bond, Y represents NR, Z represents NR′ and each of R and R′ represents, independently, hydrogen or C₁-C₆ alkyl;
(xii) when X represents CH₂—C—OR′, —(y)— represents a single bond, —(x)— represents a double bond, Y represents NR, Z represents N and each of R and R′ represents, independently, hydrogen or C₁-C₆ alkyl;
(xiii) when X represents CO—CH₂, —(y)— represents a single bond, —(x)— represents a single bond, Y represents NR′, Z represents NR and each of R and R′ represents, independently, hydrogen or C₁-C₆ alkyl; or
(xiv) when X represents R′O—C—CH₂, —(y)— represents a double bond, —(x)— represents a single bond, Y represents N, Z represents NR and each of R and R′ represents, independently, hydrogen or C₁-C₆ alkyl;
each of R₁, R₂ and R₅ represents, independently, hydrogen, halogen, hydroxy, C₁-C₆ alkoxy, C₁-C₆ acyloxy or aroyloxy;
each of R₇ and R₈ represents, independently, hydrogen or halogen;
each of R₉ and R₁₀ represents, independently, hydrogen; C₁-C₆ alkyl unsubstituted or substituted by aryl; C₁-C₆ acyl; aroyl; or R₉ and R₁₀, taken together, represent methylene;
or a pharmaceutically acceptable salt thereof.

13. A compound of formula (Ib) according to claim 10 wherein
R₆ is as defined in claim 10;
R₃ and R₄, taken together, represent a 5 or 6 membered fused ring system having the following formula
—Y—(y)—X—(x)—Z—
wherein
(ii) when X represents CR, —(y)— represents a double bond, —(x)— represents a single bond, Y represents N, Z represents NR′, oxygen (O) or sulphur (S), R represents hydrogen or C₁-C₆ alky optionally substituted with C₁-C₆ dialkylamino and R′ represents hydrogen or C₁-C₆ alkyl;
(iii) when X represents C—OR, —(y)— represents a double bond, —(x)— represents a single bond, Y represents N, Z represents NR′, oxygen (O) or sulphur (S) and each of R and R′ represents, independently, hydrogen or C₁-C₆ alkyl;
(iv) when X represents C═O, —(y)— represents a single bond, —(x)— represents a single bond, Y represents NR′, Z represents NR′, oxygen (O) or sulphur (S) and R′ represents hydrogen or C₁-C₆ alkyl;
(v) when X represents C—SR, —(y)— represents a double bond, —(x)— represents a single bond, Y represents N, Z represents NR′, oxygen (O) or sulphur (S) and each of R and R′ represents, independently, hydrogen or C₁-C₆ alkyl;
(vi) when X represents C═S, —(y)— represents a single bond, —(x)— represents a single bond, Y represents NR′, Z represents NR′, oxygen (O) or sulphur (S) and R′ represents hydrogen or C₁-C₆ alkyl;
(vii) when X represents N, —(y)— represents a double bond, —(x)— represents a single bond, Y represents N, Z represents NR′ and R′ represents hydrogen or C₁-C₆ alkyl;
(viii) when X represents O═C—C═O, —(y)— represents a single bond, —(x)— represents a single bond, Y and Z represent NR′ wherein R′ represents hydrogen or C₁-C₆ alkyl;
(ix) when X represents RO—C—C═O, —(y)— represents a double bond, —(x)— represents a single bond, Y represents N, Z represents NR′ and each of R and R′ represents, independently, hydrogen or C₁-C₆ alkyl;
(x) when X represents RO—C—C—OR, —(y)— represents a double bond, —(x)— represents a double bond, Y and Z represent N and R represents hydrogen or C₁-C₆ alkyl;
(xi) when X represents CH₂—CO, —(y)— represents a single bond, —(x)— represents a single bond, Y represents NR, Z represents NR′ and each of R and R′ represents, independently, hydrogen or C₁-C₆ alkyl;
(xii) when X represents CH₂—C—OR′, —(y)— represents a single bond, —(x)— represents a double bond, Y represents NR, Z represents N and each of R and R′ represents, independently, hydrogen or C₁-C₆ alkyl;
(xiii) when X represents CO—CH₂, —(y)— represents a single bond, —(x)— represents a single bond, Y represents NR′, Z represents NR and each of R and R′ represents, independently, hydrogen or C₁-C₆ alkyl; or
(xiv) when X represents R′O—C—CH₂, —(y)— represents a double bond, —(x)— represents a single bond, Y represents N, Z represents NR and each of R and R′ represents, independently, hydrogen or C₁-C₆ alkyl;
each of R₁, R₂ and R₅ represents, independently, hydrogen, halogen, hydroxy, C₁-C₆ alkoxy, C₁-C₆ acyloxy or aroyloxy;
each of R₇ and R₈ represents, independently, hydrogen or halogen;
each of R₉ and R₁₀ represents, independently, hydrogen; C₁-C₆ alkyl unsubstituted or substituted by aryl; C₁-C₆ acyl; aroyl; or R₉ and R₁₀, taken together, represent methylene;
or a pharmaceutically acceptable salt thereof.

14. A compound of formula (Ic) according to claim 11 wherein
R₆ is as defined in claim 11;
each of R₁, R₂ and R₅ represents, independently, hydrogen, halogen, hydroxy, C₁-C₆ alkoxy, C₁-C₆ acyloxy or aroyloxy;
each of R₇ and R₈ represents, independently, halogen, cyano, C₁-C₆ alkoxycarbonyl, carboxyl, nitro, amino, C₁-C₆ monoalkylamino C₁-C₆ dialkylamino, C₁-C₆ trialkylammonium halides, C₁-C₄ acylamino, aroylamino, C₁-C₆ alkylsulfonylamino, arylsulfonylamino, C₁-C₆ alkylaminosulfonyl, or arylaminosulfonyl, provided that R₇ and R₈ are not contemporarily hydrogen;
each of R₉ and R₁₀ represents, independently, hydrogen; C₁-C₆ alkyl unsubstituted or substituted by aryl; C₁-C₆ acyl; aroyl; or R₉ and R₁₀, taken together, represent methylene,
or a pharmaceutically acceptable salt thereof.

15. A compound selected from the group consisting of:
3-chloro-2-(3,4-dihydroxyphenyl)-7,8-dihydroxy-4H-chromen-4-one (compound 37);
3-chloro-2-(3,4-dimethoxyphenyl)-7,8-dimethoxy-4H-chromen-4-one (compound 38);
3-cyano-2-(3,4-dihydroxyphenyl)-7,8-dihydroxy-4H-chromen-4-one (compound 39);
3-cyano-2-(3,4-dimethoxyphenyl)-7,8-dimethoxy-4H-chromen-4-one (compound 40);
3-fluoro-2-(3,4-dihydroxyphenyl)-7,8-dihydroxy-4H-chromen-4-one (compound 41);
2-(3,4-dimethoxyphenyl)-3-fluoro-7,8-dimethoxy-4H-chromen-4-one (compound 42);
2-(4-fluorophenyl)-7,8-dihydroxy-4H-chromen-4-one(compound 43);
2-(3-fluorophenyl)-7,8-dihydroxy-4H-chromen-4-one(compound 44);
2-(3-chlorophenyl)-7,8-dihydroxy-4H-chromen-4-one (compound 45);
2-(3,4-dichlorophenyl)-7,8-dihydroxy-4H-chromen-4-one (compound 46);
5-chloro-2-(3,4-dihydroxyphenyl)-7,8-dihydroxy-4H-chromen-4-one (compound 47);
5-chloro-2-(3,4-dimethoxyphenyl)-7,8-dimethoxy-4H-chromen-4-one (compound 48);
6-chloro-2-(3,4-dihydroxyphenyl)-7,8-dihydroxy-4H-chromen-4-one (compound 49);
6-chloro-2-(3,4-dimethoxyphenyl)-7,8-dimethoxy-4H-chromen-4-one (compound 50);
2-(3,4-diaminophenyl)-7,8-dimethoxy-4H-chromen-4-one (compound 51);
2-(3,4-diaminophenyl)-7,8-dihydroxy-4H-chromen-4-one (compound 52);
N-[4-(7,8-dimethoxy-4-oxo-4H-chromen-2-yl)-2-nitrophenyl]acetamide (compound 53);
2-(4-acetylamino-3-nitrophenyl)-7,8-dihydroxy-4H-chromen-4-one (compound 54);
2-(4-amino-3-nitrophenyl)-7,8-dimethoxy-4H-chromen-4-one (compound 55);
2-(4-amino-3-nitrophenyl)-7,8-dihydroxy-4H-chromen-4-one (compound 56);
2-(3,4-diacetylaminophenyl)-7,8-dihydroxy-4H-chromen-4-one (compound 57);
N-[2-(acetylamino)-4-(7,8-dimethoxy-4-oxo-4H-chromen-2-yl)phenyl]acetamide (compound 58);
2-(3,4-di-trifluoroacetylaminophenyl)-7,8-dihydroxy-4H-chromen-4-one (compound 59);
2-(3,4-di-trifluoroacetylaminophenyl)-7,8-dimethoxy-4H-chromen-4-one (compound 60);
2-(1H-benzimidazol-5-yl)-7,8-dimethoxy-4H-chromen-4-one (compound 61);
2-(1H-benzimidazol-5-yl)-7,8-dihydroxy-4H-chromen-4-one (compound 62);
2-(1H-benzimidazol-5-yl)-8-hydroxy-7-methoxy-4H-chromen-4-one (compound 63);
4-(7,8-dihydroxy-4-oxo-4H-chromen-2-yl)benzoic acid (compound 64);
2-(1H-1,2,3-benzotriazol-5-yl)-7,8-dimethoxy-4H-chromen-4-one (compound 65);
2-(1H-1,2,3-benzotriazol-5-yl)-7,8-dihydroxy-4H-chromen-4-one (compound 66);
5-(7,8-dimethoxy-4-oxo-4H-chromen-2-yl)-1,3-dihydro-2H-benzimidazol-2-one (compound 67);
5-(7,8-dihydroxy-4-oxo-4H-chromen-2-yl)-1,3-dihydro-2H-benzimidazol-2-one (compound 68);
7,8-dimethoxy-2-(2-sulfanyl-1H-benzimidazol-5-yl)-4H-chromen-4-one (compound 69);
7,8-dihydroxy-2-(2-sulfanyl-1H-benzimidazol-5-yl)-4H-chromen-4-one (compound 70);
2-(2-amino-1H-benzimidazol-5-yl)-7,8-dimethoxy-4H-chromen-4-one (compound 71);
2-(2-amino-1H-benzimidazol-5-yl)-7,8-dihydroxy-4H-chromen-4-one (compound 72);
2-(1,3-benzoxazol-5-yl)-7,8-dimethoxy-4H-chromen-4-one (compound 73);
2-(1,3-benzoxazol-5-yl)-7,8-dihydroxy-4H-chromen-4-one (compound 74);
2-(1,3-benzothiazol-5-yl)-7,8-dimethoxy-4H-chromen-4-one (compound 75);
2-(1,3-benzothiazol-5-yl)-7,8-dihydroxy-4H-chromen-4-one (compound 76);
2-(2,4-dihydroxyphenyl)-7,8-dihydroxy-4H-chromen-4-one (compound 77);
8-hydroxy-7-methoxy-2-(3,4-dihydroxyphenyl)-4H-chromen-4-one (compound 78);
7,8-dihydroxy-2-(3-hydroxy-4-methoxyphenyl)-4H-chromen-4-one (compound 79);
7,8-dimethoxy-2-(3,4-dihydroxyphenyl)-4H-chromen-4-one (compound 80);
6-(7,8-dimethoxy-4-oxo-4H-chromen-2-yl)-1,4-dihydro-2,3-quinoxalinedione (compound 81);
6-(7,8-dihydroxy-4-oxo-4H-chromen-2-yl)-1,4-dihydro-2,3-quinoxalinedione (compound 82);
6-(7,8-dimethoxy-4-oxo-4H-chromen-2-yl)-3,4-dihydro-2(1H)-quinoxalinone (compound 83);
6-(7,8-dihydroxy-4-oxo-4H-chromen-2-yl)-3,4-dihydro-2(1H)-quinoxalinone (compound 84);
7-(7,8-dimethoxy-4-oxo-4H-chromen-2-yl)-3,4-dihydro-2(1H)-quinoxalinone (compound 85);
7-(7,8-dihydroxy-4-oxo-4H-chromen-2-yl)-3,4-dihydro-2(1H)-quinoxalinone (compound 86);
7,8-dimethoxy-2-(3-hydroxy-4-methoxyphenyl)-4H-chromen-4-one (compound 87);
2-(2,4-dimethoxyphenyl)-7,8-dimethoxy-4H-chromen-4-one (compound 88);
4-(7,8-dihydroxy-4-oxo-4H-chromen-2-yl)benzonitrile (compound 89);
2-(3,4-diacetoxyphenyl)-7,8-dihydroxy-4H-chromen-4-one (compound 90);
7,8-dihydroxy-2-(4-hydroxy-3-methoxyphenyl)-4H-chromen-4-one (compound 91);
2-(3,4-dihydroxyphenyl)-7-hydroxy-8-methoxy-4H-chromen-4-one (compound 92);
7-hydroxy-2-(4-hydroxy-3-methoxyphenyl)-8-methoxy-4H-chromen-4-one (compound 93);
7-hydroxy-2-(3-hydroxy-4-methoxyphenyl)-8-methoxy-4H-chromen-4-one (compound 94);
8-hydroxy-2-(3-hydroxy-4-methoxyphenyl)-7-methoxy-4H-chromen-4-one (compound 95);
8-hydroxy-2-(4-hydroxy-3-methoxyphenyl)-7-methoxy-4H-chromen-4-one (compound 96);
2-(3-hydroxy-4-methoxyphenyl)-7,8-dimethoxy-4H-chromen-4-one (compound 97);
2-(3-fluoro-4-hydroxyphenyl)-7,8-dihydroxy-4H-chromen-4-one (compound 98);
2-(4-fluoro-3-hydroxyphenyl)-7,8-dihydroxy-4H-chromen-4-one (compound 99);
2-(3-hydroxy-4-methoxyphenyl)-7,8-dimethoxy-4H-chromen-4-one (compound 100);
7,8-dimethoxy-2-(2-methyl-1H-benzimidazol-5-yl)-4H-chromen-4-one (compound 101);
7,8-dihydroxy-2-(2-methyl-1H-benzimidazol-5-yl)-4H-chromen-4-one (compound 102);
2-{2-[3-(Dimethylamino)propyl]-1H-benzimidazol-5-yl}-7,8-dimethoxy-4H-chromen-4-one (compound 103)
and, if the case, the pharmaceutically acceptable salts thereof.

16. A pharmaceutical composition which comprises as an active agent a compound of formula (Ia) as described in claim 9 or a pharmaceutically acceptable salt thereof, a compound of formula (Ib) as described in claim 10 or a pharmaceutically acceptable salt thereof or a compound of formula (Ic) as defined in claim 11 or a pharmaceutically acceptable salt thereof, and a pharmaceutically acceptable excipient.

17. A pharmaceutical composition according to claim 16, for use in the treatment of a telomerase-modulated disease.

18. A pharmaceutical composition according to claim 16, for use in the treatment of a cancer disease related to a deranged cancer cell growth mediated by telomerase enzyme activity.

19. A pharmaceutical composition according to claim 16, for use in the treatment of a cancer.
